(19) United States
(12) Patent Application Publication
(10) Pub. No.: US 2004/0234965 A1
(43) Pub. Date: Nov. 25, 2004

(54) Novel pathways involved in epigenetic memory

(76) Inventor: Robert Marshall Burgess JR., Temecula, CA (US)

Correspondence Address: Robert M. Burgees, Jr., 31082 Berkshire Lane, Temecula, CA, 92591, US

(21) Appl. No.: 10/444,737

(22) Filed: May 23, 2003

(51) Int. Cl. A61K 48/00; C12Q 1/68; C07H 21/02

(52) U.S. Cl. 435/6; 514/44; 536/23.1

(57) ABSTRACT

The molecular mechanisms by which central nervous system-specific genes are expressed only in the nervous system and repressed in other tissues remain a central issue in developmental and regulatory biology. The presently described invention outlines a dual mechanism of transcriptional downregulation in which the transcription factor REST/NRSF is a central component. The invention delineates short-term REST/NRSF-dependent active repression of neural-specific genes through the recruitment of histone deacetylases as well as long-term gene silencing through the recruitment of a novel biochemical complex which requires CpG methylation. Long-term silencing of neuronal-specific genes requires the recruitment of an associated corepressor, CoREST, that serves as a functional molecular beacon for the recruitment of molecular machinery that imposes silencing across a chromosomal interval, including transcriptional units that do not themselves contain REST/NRSF response elements.

### 1.0 FIELD OF THE INVENTION

The following invention describes the discovery and characterization of unique REST/NRSF-dependent neuronal gene silencing mechanisms within specific chromosomal regions.

### 2.0 BACKGROUND OF THE INVENTION

The regulation of gene transcription plays a crucial role in a wide variety of cellular processes including proliferation, lineage commitment and terminal differentiation. It is through the activation and repression of genetic activity that cellular identity is established both during embryonic development and postnatally. Several types of transcriptional repression exist, including short-term active repression and long-term gene silencing. An example of short-term active repression is the N—CoR-dependent downregulation of target gene transcription during erythrocyte and thymocyte development by nuclear receptors and MAD. Mice mutated for functional inactivation of N—CoR have demonstrated a release of nuclear receptor and MAD repressive activities in these systems (Jepsen et al., 2000, Cell, 102: 753-58). An example of long-term gene silencing is represented by the Polycomb Group Proteins (PcG) and their effects on Hox cluster gene transcription. It has been recently reported that these proteins modulate long-term gene silencing through histone H3 methylation specifically at lysine 27 via recruitment of an EED-EZH2 complex (Cao et al., 2002, Science, 298(5595): 1039-43). Thus in the case of HOX genes histone methylation patterns have been shown to denote the repressive status of particular genomic regions.

Previous studies have implicated long-term gene silencing as a critical component of the establishment of an epigenetic program for terminally differentiated cells (Bird, A. 2002, Genes Dev., 16(1): 6-21; Breiling et al., 2001, Nature, 412(6847): 651-5; Cosma et al., 1999, Cell, 97(3): 299-311). It is the discrete silencing of genes that are appropriate for a particular cell's function and morphology which allows for cellular propagation, viability, function and the maintenance of inheritable cell type diversity. An example of this is the silencing of neuron-specific genes in non-neuronal tissues and cell types. A large number of neuron-specific genes including ion channels, neurotransmitters and axonal guidance loci are inactive in non-neuronal cell types. Previous data have implicated the repressor protein REST/NRSF as a critical component of the repression in non-neuronal cells of loci containing a REST/NRSF DNA binding site, yet the mechanisms by which REST modulates the downregulation these genes in these cells are unknown (Schoenherr et al., 1996, Proc. Natl. Acad. Sci. U.S.A. 93:9881-9886; U.S. Pat. Nos. 5,935,811, 6,270,990 and 6,511,808 and herein incorporated by reference).

One possible mechanism by which specific loci are silenced is through the modulation of chromatin characteristics and organization, yet the biochemical and molecular transitions which occur at the level of chromatin and more particularly at the level of specific loci to drive long-term silencing remain unclear. The following described invention and technology outlines a REST/NRSF-containing novel biochemical complex that has the capacity to mediate long-term gene silencing. This silencing requires the recruitment of an associated corepressor, CoREST, that acts as a molecular beacon for the recruitment of specific machinery including methyltransferase activity that imposes silencing across a particular chromosomal interval and includes certain transcriptional units that do not themselves contain REST/NRSF response elements. In addition, it is shown that REST/NRSF may also drive active transcriptional repression through the recruitment of histone deacetylase activity to regulatory elements of specific target genes. Thus, the following invention outlines a dual mechanism and functional role for REST/NRSF in the downregulation of gene transcription.

It could not be obvious to anyone trained in the art that REST/NRSF mediated downregulation of gene transcription may denote long-term silencing and may be mediated through specific methyl DNA binding proteins which act in concert with histone methyltransferase enzymes and corepressor molecules to drive chromatin organization at discrete regions within the genome. In addition, it could not be obvious to anyone trained in the art that particular DNA binding element codes, such as those for REST/NRSF for example, exist within the regulatory regions of loci that are repressed through long term gene silencing mechanisms and that these codes may act to drive repression of adjacent yet considerably distant loci through a spreading effect. Finally, it could not be obvious to those trained in the art that the biochemical and molecular code described herein represents a potential avenue for therapeutic intervention for the ultimate treatment of cancer and neurological disorders such as Rett syndrome and Alzheimer's disease.

### 3.0 SUMMARY OF THE INVENTION

The present invention relates to the discovery and characterization of a novel biochemical and molecular pathway involved in the establishment and maintenance of eukaryotic cellular epigenetic memory. The invention also relates to the further characterization and utilization of the individual components of the described biochemical and molecular entity responsible for mediating the silencing of neural-specific genes in non-neuronal tissues and/or cell types for the purposes of identifying other realms of physiology regulated or silencing in a similar manner.

The biochemical and molecular components described in this disclosure are non-obvious and substantial additions to the general body of knowledge relating to long term gene silencing in tissues and differentiated cell types and are non-obvious and substantial additions to the general body of knowledge relating to REST/NRSF-mediated silencing of target gene activity.

The following embodiments are to be examples and not serve as limitations to the scope of the presently described invention. In one embodiment, a concept has been established whereby REST/NRSF downregulates or silences specific target gene activity through a combinatoric interaction with the corepressor CoREST and the methyl-DNA binding protein MeCP2, which in turn recruits, either directly or indirectly, the histone methyltransferase enzyme SUV39H1. Methylation of lysine residues on histone H3 by SUV39H1 initiates chromatin organization and condensation, presumably through the function of heterochromatic protein 1 (HP1), ultimately resulting in long term gene silencing. In this particular example, the target genes are neural- or neuronal-specific and are essentially repressed or silenced by the novel biochemical complex described in non-neural tissues and cell types. An example demonstrating this phenomenon is the repression of the NaChII gene in Rat-1 fibroblasts in tissue culture. A second example demonstrating this phenomenon is the NaChIII gene in Rat-1 fibroblasts in tissue culture. It has been demonstrated that repression of the transcriptional activity for this locus is dependent upon the biochemical complex and mechanisms described in the present invention. The concept describes a unique and previously uncharacterized mechanism for REST mediated repression which is dependent upon combinatoric interactions with the methyl-CpG binding protein MeCP2 and is independent of histone deacetylase (HDAC) activity. In addition, this is the first example of REST/NRSF mediated repression which involves the modification of histone tails via the addition of methyl groups by a methyltransferase enzyme. It is not obvious to those skilled in the art that REST/NRSF may act to mediate long-term repression either through direct or indirect interactions with MeCP2 or other methyl DNA binding proteins. It is also not obvious to those skilled in the art that REST/NRSF may mediate long-term repression through either direct or indirect interactions with SUV39H1 or other methyltransferase proteins. These studies outline a novel biochemical and molecular combinatoric complex which dictates long-term repression of specific loci.

Another embodiment of the present invention includes the biochemical complex formed containing all or a subset of the individual components REST/NRSF, CoREST, MeCP2 and SUV39H1. This complex is herein referred to as Complex A. It should be noted that the presently described invention is not limited to the specific components described in Complex A. It should also be noted that the individual components of Complex A may or may not interact directly. This complex has been demonstrated to be sufficient for the long-term suppression of gene activity, although other complexes with REST/NRSF which contain methyl-DNA binding proteins and/or histone methyltransferase enzymes or proteins significantly similar to these are also covered by the presently described invention. Other histone methyltransferase enzymes unrelated to SUV39H1 may also be assembled in Complex A instead of or in addition to the original components and provide enzymatic activity necessary for histone modification. In addition, the presently described invention is not limited to the identity of MeCP2 and CoREST. Other methyl-DNA binding proteins, such as, but not limited to, MBD2 and MBD3 and other co-repressor molecules may perform similar functions relating to gene silencing in similar complexes with REST or other repressor proteins.

Another embodiment of the presently described invention is the individual components of Complex A which are involved in and required for mediating long-term gene silencing.

An additional embodiment of the presently described invention is the individual process of biochemical recruitment of enzymatic activity necessary for the addition of methyl groups to histone H3 to specific regions and/or loci present in the genome.

In yet another embodiment of the present invention, it is postulated that a genomic spreading effect may occur in which genes that do not contain REST/NRSF binding sites may be silenced through the effects of Complex A, or similar complexes, recruited to the regulatory regions of loci within reasonable proximity.

An additional embodiment of the present invention includes the recruitment of histone methyltransferase activity for the specific purposes of mediating transcriptional repression by other repressor and/or corepressor proteins as well as methyl-DNA binding proteins unrelated to REST/NRSF, CoREST and MeCP2.

The invention also includes application of the defined characteristics of Complex A or similar complexes for the ultimate therapeutic treatment of human disorders such as Rett syndrome, Alzheimer's disease and cancer.

In an additional embodiment of the present invention, the protein CoREST has also been demonstrated to play a role in the repression or silencing of neural genes in non-neural cells.

In yet another embodiment of the present invention the methyl-DNA binding protein MeCP2 has been demonstrated to play a role in mediating the repression or silencing of neural genes in non-neural cells and tissues via the recruitment of a histone methyltransferase to the regulatory regions of the genes.

An additional embodiment of the present invention describes the histone methyltransferase enzyme SUV39H1 and its capacity to silence neural genes in non-neural cells and tissues. An example demonstrating this phenomenon is the repression of the NaChII gene in Rat-1 fibroblasts in tissue culture. A second example demonstrating this phenomenon is the NaChIII gene in Rat-1 fibroblasts in tissue culture. It has been demonstrated that repression of the transcriptional activity for this locus is dependent upon the biochemical complex and mechanisms described in the present invention. The concept describes a unique and previously uncharacterized mechanism for REST/NRSF mediated repression which is dependent upon combinatoric interactions with the methyl-CpG binding protein MeCP2 and is independent of histone deacetylases (HDAC) activity. In addition, this is the first example of REST/NRSF mediated repression which involves the modification of histone tails via the addition of methyl groups by a methyltransferase enzyme. It is not obvious to those skilled in the art that REST/NRSF may act to mediate long-term repression either through direct or indirect interactions with MeCP2 or other methyl DNA binding proteins. It is also not obvious to those skilled in the art that REST/NRSF may mediate long-term repression through either direct or indirect interactions with SUV39H1 or other methyltransferase proteins. These studies outline a novel biochemical and molecular combinatoric complex which dictates long-term repression of specific loci.

An additional embodiment of the presently described invention includes truncated dominant negative versions of the protein CoREST, which, when introduced into cell lines and/or tissues, have the specific capacity to abrogate wild-type CoREST function. These include, but are not limited to, the combination of SANT domains 1 and/or 2 as well as each domain alone.

Yet another embodiment of the present invention includes truncated dominant negative versions of the protein MeCP2, which, when introduced into cell lines and/or tissues, have the specific capacity to abrogate wild-type MeCP2 function. These include, but are not limited to, the MeCP2 DNA binding domain alone.

Still another embodiment of the present invention includes truncated dominant negative versions of the protein REST/NRSF, which, when introduced into cell lines and/or tissues, have the specific capacity to abrogate wild-type REST/NRSF function. These include, but are not limited to, the REST/NRSF DNA binding domain alone.

One other embodiment of the present invention relates to the suppression of cell division upon introduction of any of the dominant negative versions of REST/NRSF, CoREST, MeCP2 or other repressor and co-repressor proteins which may mediate long-term repression in a manner similar to these proteins.

Another embodiment of the present invention includes the amino acid sequences of REST/NRSF, CoREST, MeCP2 and SUV39H1 as they relate to the functionality of establishing target gene repression or silencing. The presently described invention also pertains to the utilization of amino acid sequences corresponding to REST/NRSF, CoREST, MeCP2 or SUV39H1 or truncated versions of these proteins for the design of therapeutics for the treatment of disorders.

Still another embodiment of the present invention includes the nucleotide sequences of REST/NRSF, CoREST, MeCP2 and SUV39H1 or truncated versions of these genes as they relate to the functionality of establishing target gene repression or silencing.

Another embodiment of the presently described invention relates to the identification of other histone methyltransferase enzymes which are recruited via a REST/NRSF, CoREST or MeCP2—dependent complex which act either individually or combinatorically for the specific purpose of mediating long-term repression via the ultimate and eventual modification of histones through the addition of methyl groups.

One other embodiment of the present invention relates to the discovery of other cofactors which act in a manner similar to CoREST and MeCP2 which act either individually or combinatorically for the specific purpose of mediating long-term repression via the ultimate and eventual modification of histones through the addition of methyl groups.

Another embodiment of the present invention includes the amino acid and nucleotide sequences of other repressor proteins, corepressor proteins, methyl-DNA binding proteins and histone methyltransferase enzymes unrelated to those described in the present invention which act either individually or combinatorically for the specific purpose of mediating long-term repression via the ultimate and eventual modification of histones through the addition of methyl groups.

An additional embodiment of the presently described invention includes the cells and tissues in which repression or silencing of gene activity occurs via a REST/NRSF, CoREST, MeCP2 or SUV39H1—dependent mechanism.

In yet another embodiment of the presently described invention, the nucleotide sequence of the REST/NRSF DNA binding site may be utilized in a low stringency search via molecular biology protocols for the identification of REST/NRSF-regulated genes.

Yet another embodiment of the present invention is the specific organization of REST/NRSF DNA binding elements which may be utilized to define the identification of neural or neuronal-specific genes.

In still another embodiment of the present invention, a specific organization of REST/NRSF DNA binding elements may be implemented in a search for previously unidentified neural-specific genes. This search may be bioinformatical or non-bioinformatical in nature and consist merely of scanning the genome for REST/NRSF or REST/NRSF—like DNA binding sites.

Another embodiment of the present invention is the sodium channel type II gene (NaChII), which has been demonstrated to be regulated in a REST/CoREST/MeCP2/methyltransferase-dependent manner.

An additional embodiment of the present invention are other genes which are silenced or repressed by REST/NRSF—containing biochemical complexes, an example of which is the NaChIII gene.

In yet another embodiment of the present invention, the application of a bioinformatical approach to the identification of REST/NRSF regulated genes may be implemented through the use of a specific organization of REST DNA binding elements. Said organization may include multiple consensus or near-consensus REST DNA binding sites in reasonable proximity to one another.

Yet another embodiment of the presently described invention relates to the design of chemical or nonchemical agents which interact specifically with proteins or amino acid sequences corresponding to REST/NRSF, CoREST, MeCP2 and SUV39H1 for the specific purposes of downregulating or abolishing the functions of these proteins.

Yet another embodiment of the presently described invention relates to the design of chemical or nonchemical agents which interact specifically with proteins or amino acid sequences corresponding to REST/NRSF, CoREST, MeCP2 and SUV39H1 for the specific purposes of upregulating or enhancing the functions of these proteins.

One other embodiment of the present invention is the design of chemical or nonchemical agents which interact specifically with proteins or amino acid sequences corresponding to REST/NRSF regulated target genes which may or may not be transcriptionally controlled via methylation-dependent heterochromatic organization for the purposes of abrogating target gene function.

Aother embodiment of the present invention is the design of chemical or nonchemical agents which interact specifically with proteins or amino acid sequences corresponding to REST/NRSF regulated target genes which may or may not be transcriptionally controlled via methylation-dependent heterochromatic organization for the purposes of enhancing target gene function.

### 4.0 DESCRIPTION OF THE FIGURES

FIG. 1 is a demonstration of HDAC-dependent and DNA methylation-dependent repression of neuronal-specific genes in Rat-1 fibroblasts (see text for details).

FIG. 2 is a demonstration of MeCP2 mediated transcriptional repression of the endogenous methylated NaChII gene in Rat-1 fibroblasts (see text for details).

FIG. 3 is a characterization of REST/CoREST-dependent silencing of rat chromosomal interval 3q22-32 (see text for details).

FIG. 4 is an analysis of the biochemical components associating with chromosomal interval 3q22-32 which include REST/NRSF, CoREST and MeCP2 (see text for details).

FIG. 5 is a diagrammatic model of CoREST-dependent gene silencing (see text for details).

Table 1 is a listing of representative examples of putative REST/NRSF target genes with a REST/NRSE response element adjacent to the promoter based upon bioinformatics (see text for details).

### 5.0 DETAILED DESCRIPTION OF THE INVENTION

The presently described invention outlines a dual mechanism for REST/NRSF mediated downregulation of target gene transcription. In short-term active gene repression, REST/NRSF interacts with histone deacetylase enzymes to drive the inhibition of gene expression. In long-term gene silencing, a biochemical complex, herein referred to as Complex A, acts to mediate transcriptional repression through the recruitment of methyltransferase activity to specific chromosomal intervals. The complex is defined by the cofactor CoREST, which acts as a molecular beacon for the silencing machinery, including MeCP2, SUV39H1, and HP1. Methylation of histones, in this case by the methyltransferase enzyme SUV39H1, drives chromatin nucleation and transcriptional silencing. Such silencing may be maintained across specific chromosomal intervals, including genes that do not contain REST/NRSF-binding sites.

For purposes of the present invention the term “gene” will refer to any and all regions of the genome of all organisms which code for proteins. This definition will also include all control elements directly or indirectly associated with controlling the production of mRNA from the gene.

In addition, for the purposes of the present invention the term “control element” will refer to any regulatory element which dictates, controls or modulates the production of mRNA from the corresponding gene. The production of mRNA is presumed to occur, at least in part, through the binding of transcription factors.

For the purposes of the present invention the term “transcription factor” will refer to any protein which binds directly or indirectly to a control element present within a gene and dictates, controls or modulates either the production or inhibition of production of mRNA from that particular gene.

As well, for the purposes of the present invention the term “transcriptional activator” will refer to any protein which binds either directly to a DNA control element or indirectly to a DNA control element through other proteins and activates or drives the production of mRNA from the gene corresponding to that particular control element.

For the purposes of the present invention the term “transcriptional repressor” will pertain to any protein which actively downregulates and thereby represses the production of mRNA from a gene to levels below those naturally occurring in an in vivo setting or to undetectable levels.

Also for the purposes of the present invention, the term “transcriptional modulator” will refer to any protein which dictates, controls or modulates the production of mRNA from a gene.

In addition for the purposes of the present invention, the term co-repressor” will refer to any protein which interacts either directly or indirectly with another protein or proteins to drive the repression or inactivation of the production of mRNA from a gene.

A gene will be delineated as active and therefore “expressed” when a nucleotide sequence referred to as an activating element is present within the gene or in close proximity to the gene and drives the production of detectable levels of mRNA, presumably through the actions of a transcriptional activating factor or transcriptional modulator. A gene will be delineated as not expressed and therefore repressed or silenced when mRNA cannot be detected, presumably due to the absence of control activating elements, due to the absence of transcriptional activators present on those elements, due to the presence of transcriptional repressors or due to heterochromatic condensation through DNA and/or histone methylation.

Finally, for the purposes of the present invention the term “active repression” will refer to the direct downregulation of a gene due to the presence of a silencing element within that gene or in close proximity to the gene, presumably through the binding at that particular silencing element or negative regulatory element of a transcriptional repressor. An example of a transcriptional repressor is REST/NRSF.

5.1 HDAC-dependent Repression of the Neuron-specific Gene SCG10

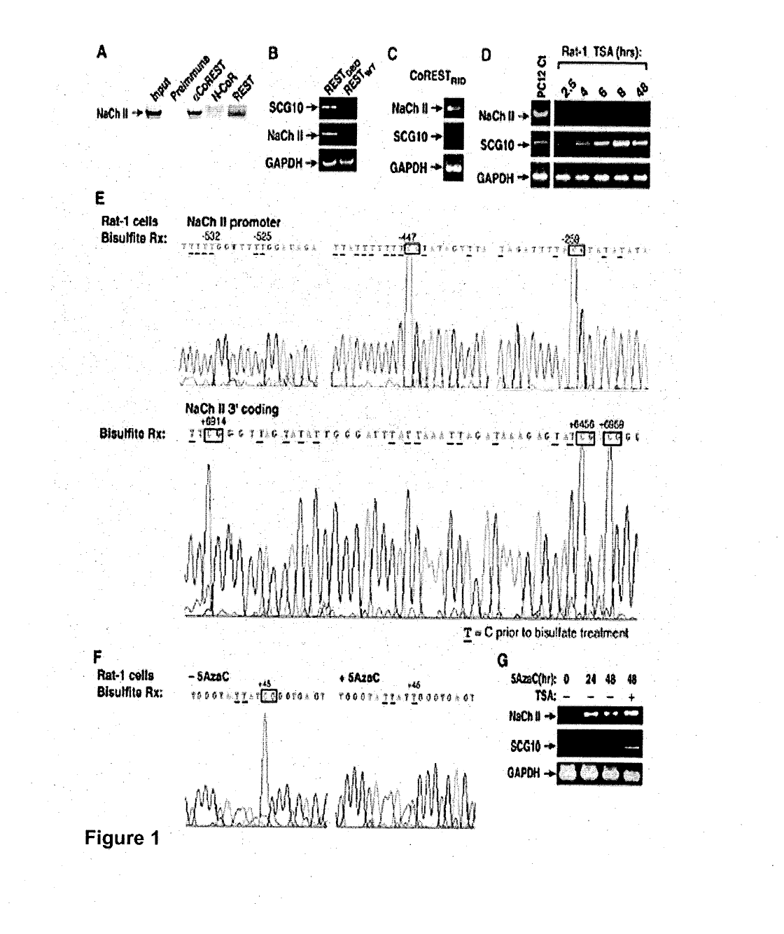

Short-term active repression plays an important role in the control of cell fate and function through the suppression of gene activity in specific cellular contexts. The deacetylation of histones has been shown to be a critical component of active gene repression. Histone deacetylase activity has been demonstrated to be recruited by transcriptional repressors for the removal of acetyl groups from histones resulting in active gene repression. A number of transcription factors capable of repression have been demonstrated to repress transcriptional activity through the recruitment of HDAC activity. Several of these include the MAD-MAX heterodimer, BCL-6 and blimp-1 (Nagy et al., 1997, Cell, 89(3): 373-80; Dhordain et al., 1998, Nucleic Acids Res., 26(20): 4645-51; Yu et al., 2000, Mol. Cell Biol., 20(7): 2592-603). REST/NRSF has been demonstrated to recruit mSin3A/HDAC1, 2 (Naruse et al., 1999, Proc. Natl. Acad. Sci. U.S.A., 96: 13691-95; Jepsen et al., 2000, Cell, 102: 753-58) but also has been shown to be in CoREST-containing complexes (You et al., 2001, Proc. Natl. Acad. Sci. U.S.A., 98: 1454-59; Humphrey et al., 2001, J. Biol. Chem., 276: 6817-22; Hakimi et al., 2002, Proc. Natl. Acad. Sci. U.S.A., 99: 7420). The presently described invention investigates the alternative roles of HDAC vs. CoREST recruitment in gene repression and/or silencing. To investigate the molecular mechanisms involved in REST/NRSF-mediated gene repression and co-repressor complexes, the neuronal-specific gene NaChII was studied and its regulation compared to that of SCG10 (Mori et al., 1990, Neuron, 4: 583-88; Ballas et al., 2001, Neuron, 31: 353-58; Naruse et al., 1999, Proc. Natl. Acad. Sci. U.S.A., 96: 13691-95; Jepsen et al., 2000, Cell, 102: 753-58). In a chromatin immunoprecipitation assay (ChIP) (Jepsen et al. 2000, Cell, 102: 753-58) from Rat-1 fibroblasts, REST/NRSF and CoREST were highly recruited to the NaChII promoter, whereas N—CoR was not (FIG. 1A). HDAC1, HDAC3 and HDAC2 were detected in small quantities or not at all in some experiments. In contrast, REST/NRSF was present on the SCG10 gene promoter with HDAC2, HDAC3 and N—CoR (Ballas et al., 2001, Neuron, 31: 353-58; Jepsen et al., 2000, Cell, 102: 753-58). Transfection of a DNA construct that encodes the REST/NRSF DNA binding domain (RESTDBD) harboring deletions of the defined NH2- and COOH-terminal repressor domains (Ballas et al., 2001, Neuron, 31: 353-58) and hence a potential dominant negative, resulted in the specific de-repression of both the SCG10 and NaChII genes (FIG. 1B). Thus, the binding of REST/NRSF functions in both establishing and maintaining repression (Schoenherr et al., 1995, Science, 267: 1360-65).

While the present invention utilizes the REST DNA binding domain as a dominant negative, it is in no way limited to this domain for the purposes of producing a dominant negative effect. Other REST/NRSF domains besides, or in addition to, the DNA binding domain may be utilized in a dominant negative fashion.

CoREST has been shown to act as a cofactor for REST/NRSF and thus its role in REST/NRSF-mediated repression of target gene transcription was characterized through a dominant negative approach. Overexpression of the REST/NRSF interaction domain of CoREST (CoRESTRID) (Ballas et al., 2001, Neuron, 31: 353-58) served as a dominant negative in Rat-1 cells and resulted in the specific derepression of the NaChII gene (FIG. 1C); in contrast, there was no effect on repression of the SCG10 gene (FIG. 1C). Thus, the present invention outlines a unique function for CoREST with respect to unique REST/NRSF target gene activity. Because CoREST can form a biochemical complex with HDAC1/2 (You et al., 2001, Proc. Natl. Acad. Sci. U.S.A., 98: 1454-59; Humphrey et al., 2001, J. Biol. Chem., 276: 6817; Hakimi et al., 2002, Proc. Natl. Acad. Sci. U.S.A., 99: 7420-25), the functional importance of HDAC's was investigated by treating Rat-1 cells with an HDAC inhibitor, trichostatin A (TSA) (300 nM) (Naruse et al., 1999, Proc. Natl. Acad. Sci. U.S.A., 96: 13691-96). When exponentially proliferating Rat-1 cells were incubated in the presence of 300 nM TSA, ectopic activation of the SCG10 gene was observed, with the maximum level of expression activity 8 hours after treatment (FIG. 1D). In contrast, even after a 48-hour treatment with TSA no detectable activation of the NaChII gene was observed (FIG. 1D). These data indicate that CoREST is selectively required to maintain NaChII but not SCG10 gene repression and outline a putative dual mechanism for transcriptional repression that switches depending upon the target gene being regulated.

While CoREST was the cofactor studied in the above experiments, the presently described invention is in no way limited to CoREST as a cofactor for REST/NRSF involved in modulating HDAC-independent transcriptional repression. Other as yet undiscovered or uncharacterized cofactors may also act in manners similar to that of CoREST and are therefore covered by the presently described invention.

Although the presently described invention focuses on the utilization of Rat-1 fibroblasts as the in vivo model system, other cell lines and tissues may be utilized and studied for the characterization and manipulation of epigenetic memory programs related to and similar to the one defined by the REST/NRSF biochemical complex. Other tissues include, but are not limited to heart, brain, spleen, lung, liver, muscle, kidney, testis, ovary, gut, hypothalamus, pituitary, tooth bud, mesoderm, ectoderm, endoderm, neural tube, somite, smooth muscle, cardiac muscle, skeletal muscle and all embryonic tissues from all possible timepoints. Other cell lines include, but are in no way limited to 13C4 (mouse/mouse, hybrid, hybridoma), 143 B (human, bone, osteosarcoma), 2 BD4 E4 K99 (mouse/mouse, hybrid, hybridoma), 3 C9-D11-H11 (mouse/mouse, hybrid, hybridoma), 3 E 1 (mouse/mouse, hybrid, hybridoma), 34-5-8 S (mouse/mouse, hybrid, hybridoma), 3T3 (mouse, Swiss albino, embryo), 3T3 L1 (mouse, Swiss albino, embryo), 3T6 (mouse, Swiss albino, embryo), 5 C 9 (mouse/mouse, hybrid, hybridoma), 5G3 (hybrid, hybridoma), 6-23 (clone 6) (rat, thyroid, medullary, carcinoma), 7 D4 (mouse/rat, hybrid, hybridoma), 72 A1 (mouse/mouse, hybrid, hybridoma), 74-11-10 (mouse/mouse, hybrid, hybridoma), 74-12-4 (mouse/mouse, hybrid, hybridoma), 74-22-15 (mouse/mouse, hybrid, hybridoma), 74-9-3 (mouse/mouse, hybrid, B cells×mycloma, hybridoma, B cell), 76-7-4 (mouse/mouse, hybrid, hybridoma), 7C2C5C12 (mouse/mouse, hybrid, B cells×myeloma, hybridoma), 9 BG 5 (mouse/mouse, hybrid, hybridoma), 9-4-3 (mouse/mouse, hybrid, hybridoma), A 172 (human, glioblastoma), A 375 (human, malignant melanoma), A 72 (dog, golden retriever, connective, not defined tumor), A-427 (human, Caucasian, lung, carcinoma), A-498 (human, kidney, carcinoma), A-704 (human, kidney, adenocarcinoma), A549 (human, lung, carcinoma), ACHN (human, Caucasian, kidney, adenocarcinoma), ACT 1 (mouse/mouse, hybrid, hybridoma), AE-1 (mouse/mouse, hybrid, hybridoma), AE-2 (mouse/mouse, hybrid, hybridoma), Aedes albopictus (mosquito—Aedes albopictus, larvae), AGS (human, Caucasian, stomach, adenocarcinoma), AK-D (cat, lung, embryonic), Amdur II (human, Caucasian, skin, fibroblast, methylmalonicacidemia), AV 3 (human, amnion), B 95.8 (monkey, marmoset, leukocyte), B-63 (mouse, mammary gland, carcinoma), B2-1 (mouse, BALB/c, embryo), B50 (rat, nervous system, nervous tissue glial tumor), B69 (mouse/mouse, hybrid, hybridoma), B95a (monkey, marmoset), BAE (bovine, aorta), BALB 3T12-3 (mouse, BALB/c, embryo), BALB 3T3 clone A31 (mouse, BALB/c, embryo), BB (fish—Ictalurus nebulosus (bullhead brown catfish), trunk), BBM.1 clone E9 (mouse/mouse, hybrid, hybridoma), BC3H1 (mouse, brain, brain tumor), BCE C/D-1b (bovine, cornea), BeWo (human, placenta, choriocarcinoma), BF-2 (fish—bluegill fry, caudal trunk), BGM (monkey, African green, kidney), BHK 21 clone 13 (hamster, golden Syrian, kidney), BNL CL.2 (mouse, BALB/c, liver, embryonic), BNL SV A.8 (mouse, liver, embryonic), BS/BEK (bovine, kidney, embryonic), BSC-1 (monkey, African green, kidney), BT (bovine, turbinate), Bu (IMR-31) (buffalo, lung), BUD-8 (human, Caucasian, skin, fibroblast), BXPC-3 (human, pancreas, adenocarcinoma), C 1271 (mouse, RIII, mammary gland, mammary tumor), C2C12 (mouse, muscle), C32 (human, melanoma, amelanotic), C6 (rat, glial tumor), Caco-2 (human, Caucasian, colon, adenocarcinoma), Caki-1 (human, Caucasian, kidney, carcinoma), Caki-2 (human, Caucasian, kidney, carcinoma), CaLu-1 (human, Caucasian, lung, carcinoma, epidermoid), Calu-3 (human, Caucasian, lung, adenocarcinoma), CAPAN 1 (human, Caucasian, pancreas, adenocarcinoma), CAPAN 2 (human, Caucasian, pancreas, carcinoma), CAR (fish—goldfish, fin), CCF-STTG1 (human, Caucasian, astrocytoma, anaplastic, grade IV), CCRF S 180 II (mouse, CFW, sarcoma), CCRF-CEM (human, Caucasian, peripheral blood, leukemia, acute lymphoblastic), CCRF-SB (human, Caucasian, peripheral blood, leukemia, acute lymphoblastic), CEM/C2 (human, leukemia, T cell), Cf2Th (dog, thymus), Chang liver (human, liver), CHO K1 (hamster, Chinese, ovary), CHP 3 (human, Black, skin, fibroblast, galactosemia), CHP 4 (human, Black, skin, fibroblast, asymptomatic galactosemia), CHSE 214 (fish—salmon, embryo), Clone 1-5c-4 WKD of Chang Conjunctiva (human, conjunctiva), Clone M-3 (mouse, (C×DBA) F1, skin, melanoma), CMT 93 (mouse, C57BL/ICRFat, rectum, carcinoma), COS-1 (monkey, African green, kidney), COS-7 (monkey, African green, kidney), CPA (bovine, endothelium, pulmonary artery), CPA 47 (bovine, endothelium, pulmonary artery), CPAE (bovine, endothelium, pulmonary artery), CRFK (cat, domestic, kidney), CRI-D11 (rat, NEDH, insulinoma), CSE 119 (fish—salmon, embryo), CV 1 (monkey, African green, kidney), CVC 7 (Agrothis segetum, hybrid, hybridoma), D 17 (dog, bone, sarcoma, osteogenic), Daudi (human, Black, lymphoma, Burkitt), DB 9 G.8 (mouse/mouse, hybrid, hybridoma), DB1-Tes (dolphin, Delphinus bairdi, testis), DeDe (hamster, Chinese, lung), Detroit 510 (human, Caucasian, skin, fibroblast, galactosemia), Detroit 525 (human, Caucasian, skin, fibroblast, Turner syndrome), Detroit 529 (human, Caucasian, skin, fibroblast, trisomy 21/Down syndrome), Detroit 532 (human, Caucasian, foreskin, trisomy 21/Down syndrome), Detroit 539 (human, Caucasian, skin, fibroblast, trisomy 21/Down syndrome), Detroit 548 (human, Caucasian, skin, fibroblast, partial D trisomy), Detroit 550 (human, skin, fibroblast), Detroit 551 (human, Caucasian, skin, embryonic), Detroit 562 (human, Caucasian, pharynx, carcinoma), Detroit 573 (human, Caucasian, skin, fibroblast, B/D translocation), Detroit 6 (human, bone marrow), DK (dog, beagle, kidney), DON (hamster, Chinese, lung), DU 145 (human, Caucasian, prostate, carcinoma), Duck embryo (duck, Pekin, embryo), E.Derm (horse, dermis), EBTr (bovine, trachea, embryonic), ECTC (bovine, thyroid, embryonic), ECV304 (human, Asiatic, umbilical cord), EIAV 12E8.1 (mouse/mouse, hybrid, hybridoma), Ep 16 (mouse/mouse, hybrid, hybridoma), EPC (fish, carp epidermal, epithelioma), EREp (rabbit, skin, embryonic), ESK-4 (pig, kidney, embryonic), FBHE (bovine, heart, embryonic), Fc 2 Lu (cat, lung, embryonic), Fc 3 Tg (cat, tongue, embryonic), FeLV 3281 (cat, lymphoma), FHM (fish—minnow, skin), FL (human, amnion), FRhK-4 (monkey, rhesus, kidney, embryonic), G-7 (mouse, Swiss-Webster, muscle), G.8 (mouse, Swiss-Webster, muscle), GCT (human, lung, metastasis, histiocytoma), GH 1 (rat, Wistar-Furth, pituitary tumor), GH 3 (rat, Wistar-Furth, pituitary tumor), Girardi heart (human, heart), GK 1.5 (mouse/rat, hybrid, hybridoma), H 16-L10-4R 5 (mouse/mouse, hybrid, hybridoma), H 9 (human, leukemia, acute lymphoblastic), H-4-II-E (rat, liver, hepatoma), H4 (human, Caucasian, brain, nervous tissue glial tumor), H4-II-E-C3 (rat, AxC, liver, hepatoma), H4TG (rat, liver, hepatoma), H9c2(2-1) (rat, BDIX, heart), Hak (hamster, Syrian, kidney), HCT 116 (human, colon, carcinoma), HCT-8 (human, intestine, ileocecal, adenocarcinoma), HEL 299 (human, Caucasian, lung, embryonic), HeLa (human, Black, cervix, carcinoma, epitheloid), HeLa 229 (human, Black, cervix, carcinoma, epitheloid), HeLa S 3 (human, Black, cervix, carcinoma, epitheloid), Hep 2 (human, Caucasian, larynx, carcinoma, epidermoid), Hep 3B2.1-7 (human, liver, carcinoma, hepatocellular), Hep G2 (human, Caucasian, liver, carcinoma, hepatocellular), Hepa 1-6 (mouse, liver, hepatoma), HFL (human, lung), HG 261 (human, Caucasian, skin, fibroblast, Fanconi anemia), HGF 24 (human, gingival stroma), HL 60 (human, Caucasian, peripheral blood, leukemia), HOS (human, Caucasian, bone, osteosarcoma), HRT 18 (human, rectum-anus, adenocarcinoma), Hs 683 (human, neuroglia, glioma), Hs 863.T (human, bone, sarcoma, Ewing's), HS 883.T (human, bone, giant cell, sarcoma), HS 888 Lu (human, Caucasian, lung), Hs-27 (human, foreskin), HSDM1C1 (mouse, Swiss albino, fibrosarcoma), HT 1080 (human, Caucasian, acetabulum, fibrosarcoma), HT 1376 (human, Caucasian, bladder, carcinoma), HT-29 (human, Caucasian, colon, adenocarcinoma), HuTu 80 (human, adenocarcinoma), I 10 (mouse, BALB/cJ, testis, Leydig cells, testicular tumor), IB-RS-2 (pig, kidney), IBRS-2 D10 (pig, kidney), IEC-6 (rat, intestine, small), IM-9 (human, Caucasian, bone marrow, multiple myeloma), IMR 31 Bu (buffalo, lung), IMR 32 (human, Caucasian, neuroblastoma), IMR-90 (human, Caucasian, lung, embryonic), Intestine 407 (human, Caucasian, intestine, embryonic), J 111 (human, leukemia, monocytic), J 774A.1 (mouse, BALB/c, monocyte-macrophage, not defined tumor), Jensen sarcoma (rat, sarcoma), JH 4 clone 1 (guinea pig, strain 13, lung), Jiyoye (human, Black, ascitic fluid, lymphoma, Burkitt), JM (human, leukemia, T cell), Jurkat J6 (human, leukemia, T cell), K 562 (human, Caucasian, pleural effusion, leukemia, chronic myeloid), KATO III° (human, Mongoloid, stomach, carcinoma), KB (human, Caucasian, mouth, carcinoma, squamous cell), KHOS/NP (human, Caucasian, bone, osteosarcoma), KMP (mouse), L 1210 (mouse, ascitic fluid, leukemia, lymphocytic), L 132 (human, lung, embryonic), L 21.6 (mouse, hybrid, hybridoma), L 243 (mouse/mouse, hybrid, hybridoma), L 5.1 (mouse/mouse, hybrid, hybridoma), L 929 (mouse, C3H/An, connective), L6 (rat, skeletal muscle), LC 540 (rat, Fisher, testis, Leydig cells, testicular tumor), LLC-MK2 (monkey, rhesus, kidney), LLC-PK1 (pig, kidney), LLC-RK1 (rabbit, New Zealand white, kidney), LLC-WRC 256 (rat, Walker, carcinoma), LM from NCTC clone 929 (mouse, C3H/An, connective), LM TK negative (mouse, C3H/An, connective), LNCaP.FGC (human, Caucasian, prostate, carcinoma), LS 180 (human, Caucasian, colon, adenocarcinoma), M 1 (mouse, SL, bone marrow, leukemia, myeloid), M-2E6 (mouse/mouse, hybrid, hybridoma), M2-1C6-4R3 (mouse/mouse, hybrid, hybridoma), MA 104 (monkey, African green, kidney, embryonic), mAB 35 (mouse/rat, hybrid, B cells×myeloma, hybridoma, B cell), MARC 145 (monkey, kidney), Mc Coy (mouse), MC/CAR (human, plasmacytoma, B cell), MCF 7 (human, Caucasian, breast, adenocarcinoma), MDBK (bovine, kidney), MDBK(BU 100) (bovine, kidney), MDCC MSB1 (chicken, avian, spleen, lymphoma), MDCK (dog, cocker spaniel, kidney), MDOK (sheep, kidney), MDTC RP 19 (turkey, lymphocyte, Marek's disease), MEL III (monkey, rhesus, mammary gland, mammary tumor), MG-63 (human, bone, osteosarcoma), MH 1 C 1 (rat, buffalo, liver, hepatoma), MH-S (mouse, lung), MIA PaCa-2 (human, Caucasian, pancreas, carcinoma), MiCl1 (mustela vison (mink), lung), MK-D6 (mouse/mouse, hybrid, hybridoma), MLA 144 (gibbon, lymphosarcoma), MOLT-3 (human, peripheral blood, leukemia, acute lymphoblastic T cell), MOLT-4 (human, peripheral blood, leukemia), MPC-11 (mouse, BALB/c, myeloma), MPK (minipig, kidney), MRC 5 (human, lung, embryonic), MRSS-1 (mouse/mouse, hybrid, hybridoma, B cell), MS (monkey), Mv 1 Lu (mustela vison (mink), lung), MVPK-1 (pig, kidney), NA C 1300 clone (mouse, brain, neuroblastoma), Namalwa (human, Black, lymphoma, Burkitt), NCTC 2544 (human, skin, keratinocyte), NCTC clone 3526 (monkey, rhesus, kidney), Neuro-2a (mouse, albino, neuroblastoma), NIH:OVCAR-3 (human, Caucasian, adenocarcinoma, ovary), NOR 10 (mouse, muscle), NRK 49F (rat, kidney), NSO (mouse, BALB/c, myeloma), OA1 (sheep, brain), OHH1.K (deer, kidney), OKT 3 (mouse/mouse, hybrid, hybridoma), OKT 4 (mouse/mouse, hybrid, hybridoma), OKT 8 (mouse/mouse, hybrid, hybridoma), P 3 HR 1 (human, lymphoma, Burkitt), P3 88 D1 (mouse, DBA/2, monocyte-macrophage, lymphoma), P3 NS1 Ag4 (mouse, myeloma), P3NP/PFN (mouse/mouse, hybrid, hybridoma), P815 (mouse, mastocytoma), PANC-1 (human, Caucasian, pancreas, carcinoma), PC 61-5-3 (mouse/rat, hybrid, hybridoma), PC-12 (rat, adrenal medulla, pheochromocytoma), PD 5 (pig, kidney), PEG 1-6 (mouse/mouse, hybrid, B cells×myeloma, hybridoma, B cell), PK 15 (pig, kidney), PLC/PRF/5 (human, liver, hepatoma, Alexander cells), Pt K1 (marsupial—potoroo, kidney), QT 35 (quail, Japanese, fibrosarcoma), QT 6 (quail, Japanese, fibrosarcoma), R 2 C (rat, Wistar-Furth, testis, Leydig cells, testicular tumor), R 9 ab (rabbit, New Zealand white, lung), R D (human, Caucasian, muscle, rhabdomyosarcoma, embryonal), R63 (mouse/mouse, hybrid, B cells×myeloma, hybridoma, B cell), RAB-9 (rabbit, New Zealand white, skin, fibroblast), Raji (human, Black, lymphoma, Burkitt), RBL 1 (rat, leukemia, basophilic), RFL 6 (rat, Sprague-Dawley, lung), RK 13 (rabbit, kidney), RK 13/1 (rabbit, kidney), RPMI 1788 (human, Caucasian, peripheral blood), RPMI 1846 (hamster, golden Syrian, skin, melanoma, melanotic), RPMI 2650 (human, nasal septum, carcinoma, squamous cell), RPMI 8226 (human, peripheral blood, myeloma), RR 1022 (rat, Amsterdam, sarcoma), RTG 2 (fish—trout, rainbow, gonad), RTO (fish—trout, rainbow, ovary), Saos-2 (human, Caucasian, bone, osteosarcoma), Sf 1 Ep (rabbit, domestic, epidermis), SIRC (rabbit, cornea), SK-LU-1 (human, Caucasian, lung, adenocarcinoma, grade III), SK-MES-1 (human, lung, carcinoma, squamous cell), SK-NEP-1 (human, Caucasian, kidney, Wilms' tumor), SK-OV-3 (human, Caucasian, ovary, adenocarcinoma), SSE 5 (fish—trout, embryo), STO (mouse, SIM, embryo), SV-T2 (mouse, BALB/c, embryo), SW 13 (human, Caucasian, adrenal cortex, adenocarcinoma), T 98 G (human, Caucasian, glioblastoma), Th 1 Lu (bat, lung), TE 671 (human, Caucasian, medulloblastoma), TK TS 13 (hamster, Syrian, kidney), U 937 (human, Caucasian, pleural effusion, lymphoma, histiocytic), VERO (monkey, African green, kidney), VERO 76 (monkey, African green, kidney), VERO C 1008 (monkey, African green, kidney), WC 1 (fish, dermis, sarcoma), WF 2 (fish—Walley whole fry, fibroblast), WI 26 VA 4 (human, Caucasian, lung, embryonic), WI 38 (human, Caucasian, lung, embryonic), WI 38 VA 13 (human, Caucasian, lung, embryonic), WI-1003 (human, lung), WISH (human, amnion), WM 115 (human, skin, melanoma), XC (rat, Wistar, sarcoma), Y 1 (mouse, LAF1, adrenal cortex, adrenal tumor), ZR-75-1 (human, Caucasian, breast, carcinoma) and any other as yet undiscovered or uncharacterized cell lines through which the presently described invention relating to REST/NRSF repressive capacities may be studied, characterized or manipulated.

In the presently described invention REST/NRSF is demonstrated to mediate short-term active repression through the recruitment of histone deacetylase (HDAC) activity. HDAC's which may be recruited by REST/NRSF to mediate short-term active repression include, but are in no way limited to, HDAC1, HDAC2, HDAC4, HDAC5, HDAC6, HDAC7, HDAC8 and any other as yet undiscovered or uncharacterized proteins which effectively modify chromatin.

5.2 CpG Methylation is Required for Silencing NaChII Gene Transcription

The methylation of CpG islands plays a broad role in the silencing of transcriptional activity. The CpG methylation status of a particular locus directly affects the activity of that gene (Razin et al., 1989, Biochim. Biophys. Acta, 782: 331-36). Unmethylated CpG islands allow for transcriptional activation of the gene under allowable circumstances while widespread methylation of CpG's results in gene silencing. The presently described invention investigated the role of CpG methylation in REST/NRSF-mediated transcriptional silencing. Because TSA failed to reduce NaChII gene repression and because DNA methylation is a widely used strategy in gene silencing (Razin et al., 1989, Biochim. Biophys. Acta, 782: 331-36), the CpG methylation status of the NaChII gene in Rat-1 cells was examined. Within the genome, from 60 to 90% of the cytosine methylation occurs at CpG dinucleotides (Razin et al., 1989, Biochim. Biophys. Acta, 782: 331-36; Tweedie et al., 1997, Mol. Cell. Biol., 17: 1469-74; Brandies et al., 1993, Bioassays, 15: 709-14). With the use of the sodium bisulfite genomic-modification sequencing approach, it was found that the NaChII promoter region exhibited a sparse pattern of CpG methylation (CmpG), with three sites (−447, −259 and +45) preferentially methylated, whereas the CpGs further along the 3′ end of the gene exhibited a more robust methylated CpG pattern (FIG. 1E). Treatment of Rat-1 cells with 5′-aza′cytidine (5AzaC) for a prolonged period of time (up to 72 hours) to reverse DNA methylation reduced specific CpG methylation in the NaChII gene promoter (FIG. 1F) and caused derepression of the NaChII but not the SCG1-gene (FIG. 1G). These data suggest that the NaChII gene might be silenced in a CmpG-dependent manner.

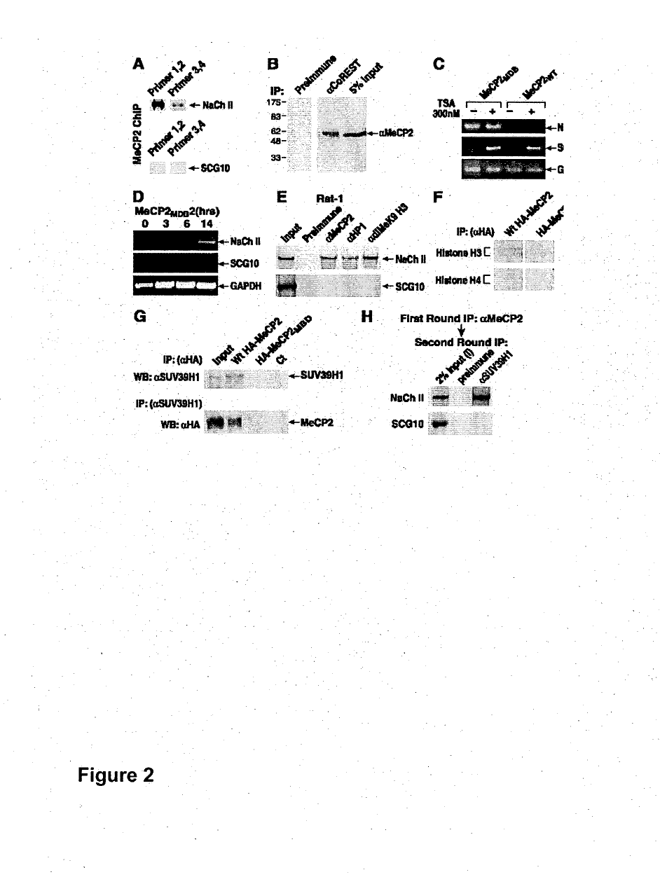

Among the many proteins that bind to methylated DNA, MeCP2 characteristically binds to single, symmetrical CmpG pairs in any sequence context (Tate et al., 1993, Curr. Opin. Genet. Dev., 3: 226-231; Siegfried et al., 1999, Nature Genet., 22: 203-208; Lewis et al., 1992, Cell, 69: 905) and has been functionally linked to gene silencing (Lorincz et al., 2001, Mol. Cell Biol., 21: 7913-18; Amir et al., 1999, Nature Genet., 23:185-90; Chen et al., 2001, Nature Genet., 27: 327-332; Bell et al., 2000, Nature, 405:482-87). Because it is also robustly expressed in Rat-1 cells, the possible participation of MeCP2 in NaChII gene repression was investigated. ChIPs were performed on Rat-1 cells using an MeCP2-specific immunoglobulin G (IgG) (FIG. 2A) and primers from the REST-binding element in the promoters as well as from the 3′ coding regions of SCG10 and NaChII genes. MeCP2 was present in both the promoter and exon and intron regions of the NaChII gene but did not bind to the SCG10 gene (FIG. 2A). No detectable quantities of the methyl DNA binding proteins MBD3 or MBD4 were observed on the NaChII gene promoter. Given the recruitment of both MeCP2 and CoREST to the NaChII gene promoter, the biochemical relationship between MeCP2 and CoREST was also investigated. An affinity-purified polyclonal CoREST antibody was used in immunoprecipitation assays and detected either direct or indirect interactions between endogenous MeCP2 and CoREST in Rat-1 cells (FIG. 2B). Thus the present invention outlines a biochemical interaction, either direct or indirect, between CoREST and MeCP2. The overexpression of the DNA binding domain of MeCP2 (MeCP2MDB) as a putative dominant negative resulted in derepression of the NaChII gene, but had no effect on repression of the SCG10 gene (FIG. 2C). The effects of MeCP2MDB on derepression of NaChII gene transcription after >12 hours in synchronized Rat-1 cells suggest that it blocks reestablishment of the repression apparatus after DNA replication (FIG. 2D). Although the class I HDACs were shown to interact with MeCP2 via mSin3 (18), MeCP2 is preferentially localized to pericentromeric heterochromatin (Lewis et al., 1992, Cell, 69: 905-10), a region of the highest 5′-CmpG concentration (Lorincz et al., 2001, Mol. Cell Biol., 21: 7913-18), suggesting that some components of heterochromatic establishment may use MeCP2 as a readout for long-term repression. Therefore, MeCP2 as well as REST/NRSF, CoREST, and DNA methylation is required in establishing and maintaining TSA-independent repression of the NaChII gene in Rat-1 cells. The present invention therefore outlines a biochemical complex, herein referred to as Complex A, including REST/NRSF, CoREST and a DNA methylation activity required for establishment and maintenance of long-term gene silencing. It should be noted that these biochemical components may interact directly or indirectly for the ultimate purpose of long-term gene silencing.

ChIP analysis was applied to determine whether similar events that led to the formation of silenced regions at the centromere, at mating-type loci, and during X chromosome inactivation (Gregory et al., 2001, Mol. Cell. Biol., 21: 5426-31; Heard et al., 2001, Cell, 107: 727-32; Rea et al., 2000, Nature, 406: 593-98; Mechtler et al., 2000, Nature, 406: 593-98; Rice et al., 2001, Curr. Opin. Cell Biol., 13: 263-68; Noma et al., 2001, Science, 293: 1150-55; Bannister et al., 2001, Nature, 410: 120-25; Litt et al., 2001, Science, 293: 2453-58; Lachner et al., 2001, Nature, 410: 116-121) pertain to REST/CoREST-dependent gene silencing. These experiments revealed the presence of heterochromatic protein 1 (HP1) as well as MeCP2 (FIG. 2E) and CoREST (FIG. 1A) on the NaChII promoter. HP1 interacts with a specially modified histone H3 (dimethyl Km9 histone H3) and is proposed to cause spreading of heterochromatic regions at the B-globin locus and in X chromosome inactivation (Kokura et al., 2001, J. Biol. Chem. 276:34115-20; Gregoryet al., 2001, Mol. Cell Biol., 21: 5426-31; Heard et al., 2001, Cell, 107: 727-32). Dimethyl Km9 histone H3 was observed on the NaChII but not the SCG10 promoter (FIG. 2E). These data indicate the possibility that a spreading effect occurs in chromosomal regions regulated by REST/NRSF and CoREST/MeCP2-dependent mechanisms.

While HP1 interacts with the NaChII promoter, other heterochromatic proteins related or unrelated to HP1 may also interact with the NaChII promoter or the promoters of other REST/NRSF target genes to cause a spreading effect with respect to transcriptional silencing and are therefore covered by the present invention.

Immunoprecipitation from Rat-1 cells of hemagglutinin (HA)-tagged holo-MeCP2 or HA-tagged MeCP2DBD revealed histone H3- but not histone H4-specific methyltransferase activity in the immunoprecipitated complex containing holo-MeCP2 (FIG. 2F). In contrast, no methyltransferase activity was recovered from immunoprecipitated complexes associated with the HA-tagged dominant-negative form of MeCP2 (MeCP2DBD) (FIG. 2F).

The presently described invention also investigated the identity of the methyltransferase putatively involved in histone H3 methylation during REST/NRSF-dependent long-term gene silencing. Mammalian histone lysine methyltransferase suppressor of variegation 39H1 (SUV39H1) initiates silencing with selective methylation of Lys9 of histone H3, thus creating a high-affinity binding site for HP1 (Lachner et al., 2001, Nature, 410: 116-121; Nakayama et al., 2000, Cell, 101: 307-12; Nakayama et al., 2001, Science, 292: 10-14; U.S. Pat. Nos. 6,555,329 and 6,184,211 herein incorporated by reference). When an antibody to endogenous SUV39H1 was used for immunoprecipitation, MeCP2 was effectively co-immunoprecipitated; conversely, anti-HA antibodies to HA-tagged MeCP2 could immunoprecipitate SUV39H1 (FIG. 2G). Two consecutive rounds of immunoprecipitation for the ChIP showed that MeCP2 and SUV39H1 (FIG. 2H) and CoREST and MeCP2 were present on the same NaChII transcription units (FIG. 2I). Further, MeCP2 was selectively immunoprecipitated from mixed histones prepared from Rat-1 cell nuclei by anti-di-Me K9 histone H3, but not antiAcK14 histone H3 or antiP10 H3 histone IgG's. Thus MeCP2 and SUV39H1 form a biochemical interaction, either direct or indirect, within cells and it is postulated that it is the methyltransferase activity of SUV39H1 which is responsible for histone H3 modification. It is the combination of SUV39H1 with REST/NRSF, CoREST and MeCP2 that defines a biochemical complex, referred to herein as Complex A, necessary for the effective long-term gene silencing of the NaChII locus. While the presently described invention indicates SUV39H1 as at least one methyltransferase activity involved in histone H3 modification and subsequent transcriptional silencing, it is in no way limited to this particular methyltransferase. Other methyltransferase enzymes, either known or as yet undiscovered, may also play roles in the gene silencing function of REST/NRSF-containing complexes.

While the presently described invention delineates a specific biochemical complex, herein referred to as Complex A, for the initiation and maintenance of long-term gene silencing, it is in no way limited to the individual components of this complex. For example, MeCP2 is defined as the methyl DNA binding protein responsible for Complex A recruitment to methylated DNA. Other methyl DNA binding proteins may also be involved and/or sufficient for recruitment to methyl DNA regions. These include, but are not limited to, MeCP1, MBD1, MBD2, MBD3 and MBD4. As mentioned above, other enzymes exhibiting methyltransferase activity may also be involved in Complex A. Finally, other cofactors related or unrelated to CoREST may also be involved in Complex A and each of which therefore is covered by the presently described invention.

5.3 CoREST-dependent Silencing of a Chromosomal Region

Bioinformatics-based genome-wide searches for the presence or absence of particular nucleotide sequences has been shown to be an indispensable tool in the discovery and annotation of both coding and noncoding gene sequence information at the global level (Mieda et al., 1997, J. Biol. Chem., 272: 5854-59). To identify previously unknown REST/NRSF target genes (Mori et al., 1990, Neuron, 583-88), a genome-wide search for REST/NRSF binding sites on the basis of the consensus site derived from experimentally confirmed RE1/NRSF with four invariant residues critical for function permitting four mismatches was performed (Pevzner et al., 2000, Intelligent Systems in Molecular Biology, AAAI Press, La Jolla, Calif., 269-78). This bioinformatics approach revealed 1047 potential REST-binding sites in the genome, all but 40 located adjacent (+−2kb) to known or predicted genes. While the presently described invention outlines 1047 potential REST/NRSF binding sites utilizing a search tool denoting four invariant residues, other more variant REST/NRSF binding sites may also recruit REST/NRSF-containing complexes and are therefore covered by the presently described invention.

Many of the putative REST/NRSF target genes have sufficiently well-characterized expression patterns, suggesting that around 90% can be assigned as strictly or predominantly neural-specific. The predicted genes encode a wide variety of functional molecules including ligands; ion channels; receptors; receptor-associated factors; and cytoskeletal and adhesion molecule-factors involved in axonal guidance, transport machinery, transcription factors, and cofactors; a portion of which are listed in Table 1 and are covered by the presently described invention. However, some genes are not neuronal-specific, including a cohort of genes involved in angiogenesis and chromatin remodeling. In addition, while the presently described invention outlines a compendium of REST/NRSF-regulated target genes via the described bioinformatics approach (Table 1), it is in no way limited to these target genes. Other as yet unidentified REST/NRSF-dependent target genes may also be identified via a REST/NRSF binding site bioinformatics or manual searching approach and are therefore covered by the presently described invention.

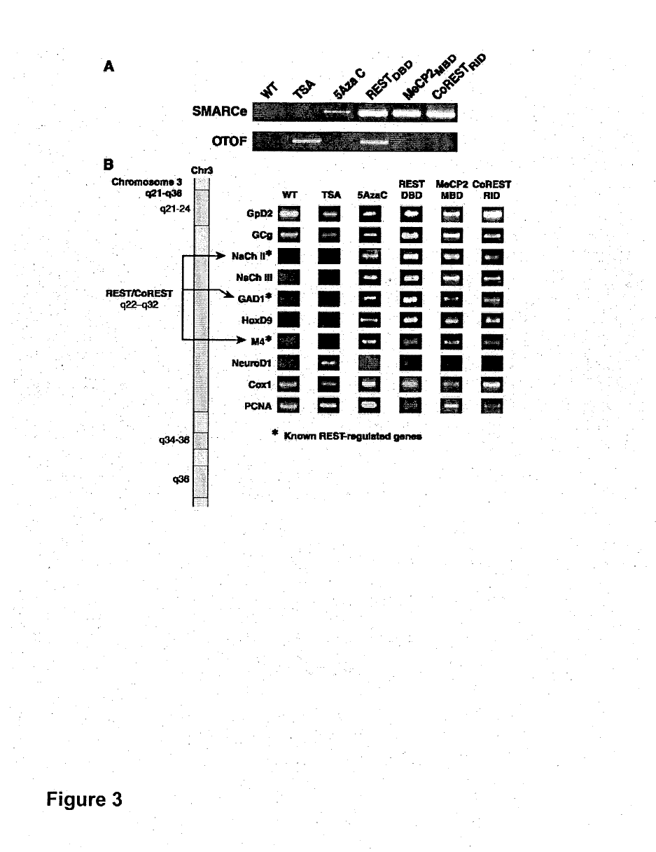

Evaluation of the effects of TSA and 5AzaC on several predicted REST/NRSF target genes suggests that there will be numerous genes exhibiting REST/NRSF-dependent silencing that require DNA methylation (e.g. SMARCe), as well as genes exhibiting HDAC-dependent repression mediated by REST/NRSF (e.g. OTOF) (Pevzner et al., 2000, Intelligent Systems in Molecular Biology, AAAI Press, La Jolla, Calif., 269-78). Both genes require REST for their repression, but overexpression of CoRESTRID or MeCP2MBD causes derepression of SMARCe in Rat-1 cells (FIG. 3A), whereas OTOF is reactivated only in the TSA-challenged Rat-1 cells (FIG. 3A). Therefore, the presently described invention outlines REST/NRSF-dependent transcriptional repression mechanisms which are unique to the particular loci being repressed.

The search for REST/NRSF binding sites revealed that many putative REST-regulated genes were tightly clustered. It was found that several neuronal-specific genes grouped together in the rat genomic interval 3q22-32 where the NaChII gene is mapped. In this interval, the only RE1/NRSE elements identified by informatics were in the promoters of the REST/NRSF-regulated neuronal-specific NaChII, GAD1 and M4 (Mieda et al., 1997, J. Biol. Chem., 272: 5854; Wood et al., 1996, J. Biol. Chem., 271:14221-26). Although eight sodium channel genes are organized in a cluster that mapped to the corresponding chromosome 2 interval in the human genome, current information permits only the NaChIII gene to be clearly mapped to the rat chromosome 3q22-32 interval. The HoxD9 gene mapped to the same interval (3q24-32), and neither NaChIII nor HoxD9 contained REST/NRSF sites. Such grouping of REST-regulated genes at the rat locus 3q22-32 raised the question of whether CoREST/MeCP2-mediated silencing is imposed on other REST-regulated genes in the interval (such as GAD1 and M4) and whether a similar mode of repression can be extended to genes not harboring REST response elements (such as NaChIII and HoxD9).

Data from transcriptional expression profiling in Rat-1 cells demonstrated that NaChII, NaChIII, GAD1, HoxD9, M4, and NeuroD1 were not expressed in Rat-1 cells (FIG. 3B), whereas genes 5′ (such as GpD2 and GCg) or 3′ (such as Cox1 and PCNA) to the interval 3q22-32 were highly expressed. Treatment of Rat-1 cells with TSA did not alter the basal level of expression of genes within the putative REST/NRSF-dependent gene interval but did cause activation of the NeuroD1 gene (FIG. 3B). However, a derepression of all tested genes in the interval flanked by the NaChII and M4 REST/NRSF target genes was observed when cells were treated with 5AzaC (FIG. 3B). Thus, the present invention suggests that the chromosomal region 3q22-32 between the NaChII and the M4 genes is silenced in a DNA methylation-dependent fashion, with the REST-regulated genes potentially serving as organizers of the silent interval. Consistent with this model, NaChII, NaChIII, GAD1, HoxD9, and M4 were all reactivated by overexpression of the RESTDBD, CoRESTRID, or MeCP2MDB dominant-negative factors (FIG. 3B). However, no reactivation of the transcriptional activity of another repressed gene, NeuroD1, was observed in the same experiments. Therefore, the presently described invention delineates a specific chromosomal interval in which genes that contain REST/NRSF binding sites as well as those that do not are repressed through long-term gene silencing mechanisms via a spreading effect. While the interval 3q22-32 appears to exhibit widespread REST/NRSF-mediated transcriptional silencing, the presently described invention is in no way limited to this particular interval or the genes located within this interval. Other chromosomal intervals may also be regulated in a REST/NRSF-dependent fashion whereby a spreading effect of transcriptional silencing occurs.

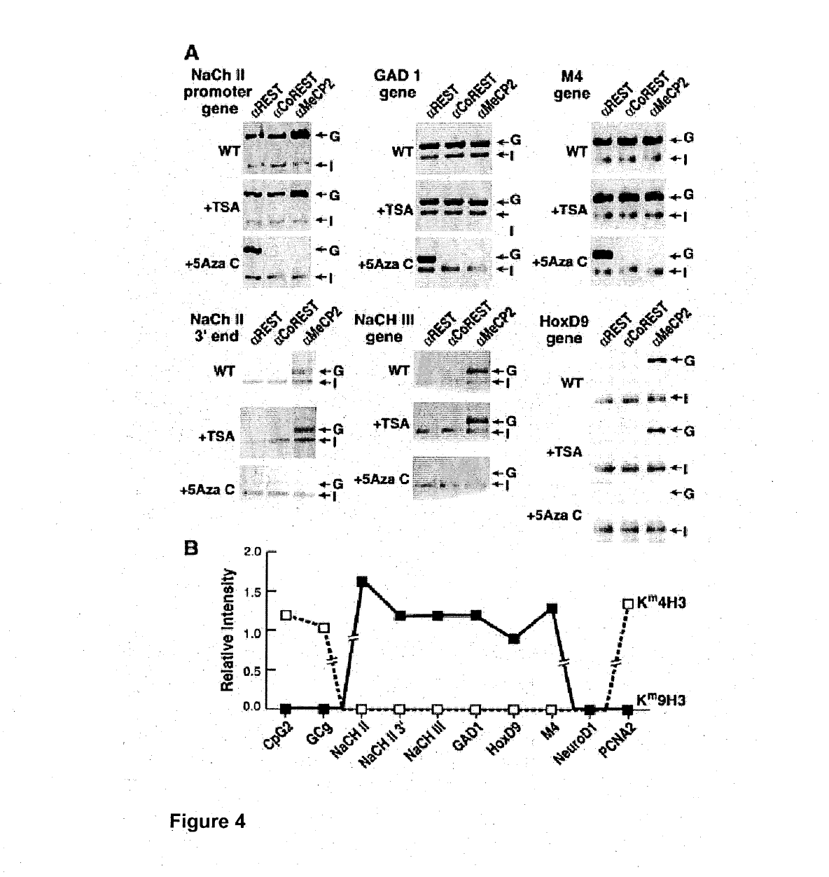

The invention also outlines the presence or absence of REST/NRSF and CoREST on different target genes in Rat-1 cells. ChIP analysis was performed with the use of the polymerase chain reaction (PCR) with an internal standard (Jenuwein et al., 2001, Science, 293: 1074-79) to normalize the results from the different genomic segments of NaChII, NaChIII, GAD1, HoxD9, and M4 gene promoters, as well as a 3′ coding region of NaChII (FIG. 4A). The results indicated that REST/NRSF and CoREST were associated only with the promoters of REST/NRSF-regulated genes (NaChII, GAD1, M4) and not detected on the 3′ coding region of NaChII (FIG. 4A). The results indicated that REST/NRSF and CoREST were associated only with the promoters of REST/NRSF-regulated genes (NaChII, GAD1, M4) and not detected on the 3′ end of NaChII gene or on the promoters of NaChIII or HoxD9 genes (FIG. 4A). This is consistent with predictions of the bioinformatics search. In contrast, MeCP2 was present throughout the interval on the six tested genomic segments. Thus, although treatment with TSA did not substantially reduce the degree of MeCP2 occupancy on the promoters of these genes, the genomic demethylation consequent to 5AzaC treatment was associated with release of MeCP2 from all of the segments within the interval (FIG. 4A). These data outline the identities of Complex A components on particular REST/NRSF target genes and describes the biochemical mechanisms by with HDAC inhibition vs. demethylation affects component occupancy on promoters.

Whereas REST/NRSF association with promoters of these genes was observed regardless of methylation status (FIG. 4A), CpG methylation appeared to be required for REST/NRSF/CoREST complex formation on the promoters of NaChII, GAD1, and M4 genes, because binding of CoREST was eliminated after treatment with 5AzaC (FIGS. 4A). Treatment with TSA had no functional effect on the repression status of the genes in the interval (FIG. 3B) and did not alter the associations of CoREST and MeCP2 proteins with tested genes (FIGS. 3B and 4A).

These data are consistent with a model whereby the presence of a REST-CoREST complex on specific promoters nucleates a progressive silencing across the interval, perhaps by sequestering the methylation-modified chromatin to an inactive nuclear matrix to allow silencing to spread across the interval. Because the “histone code” serves critical epigenetic aspects of transcriptional control and the correlation of specific histone H3 modifications in silencing of the globin locus, X chromosome inactivation, and the MHL loci in yeast (Chen et al., 2001, Nature Genet., 27: 327-32; Heard et al., 2001, Cell, 107: 727-32; Bannister et al., 2001, Nature, 410: 120-24), the presence of Km9 histone H3 and Km4 histone H3 across the interval was evaluated by ChIP analysis, with internal standard segments. All known gene targets in the rat Ch3 q22-32 interval contained dimethyl K9 histone H3 but not methyl K4 histone H3 (FIG. 4B). This correlation is similar to that observed for the silenced B globin locus (Litt et al., 2001, Science, 293: 2453-58).

5.4 Co-repressor-dependent Silencing by REST/NRSF

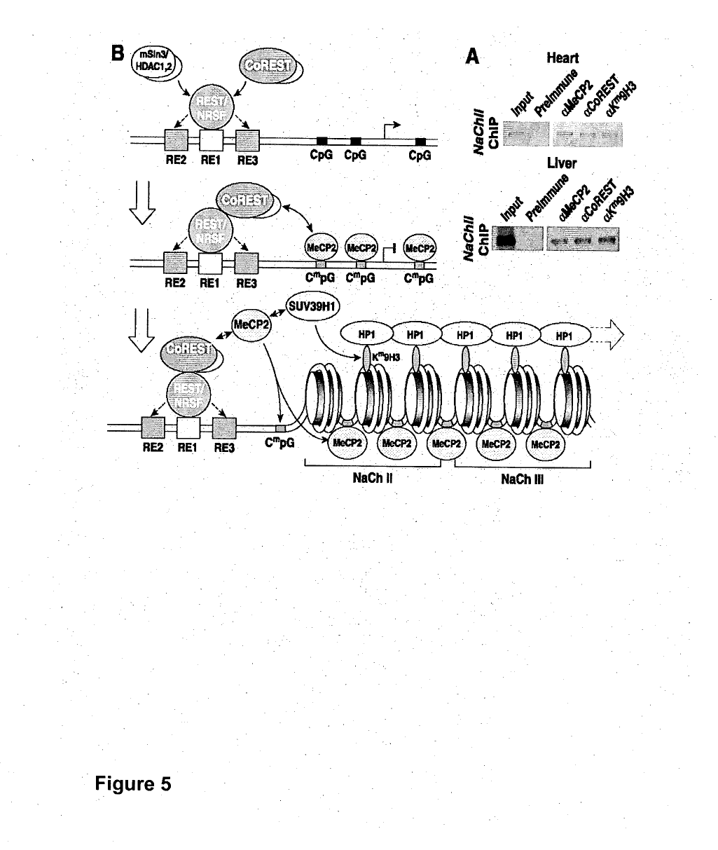

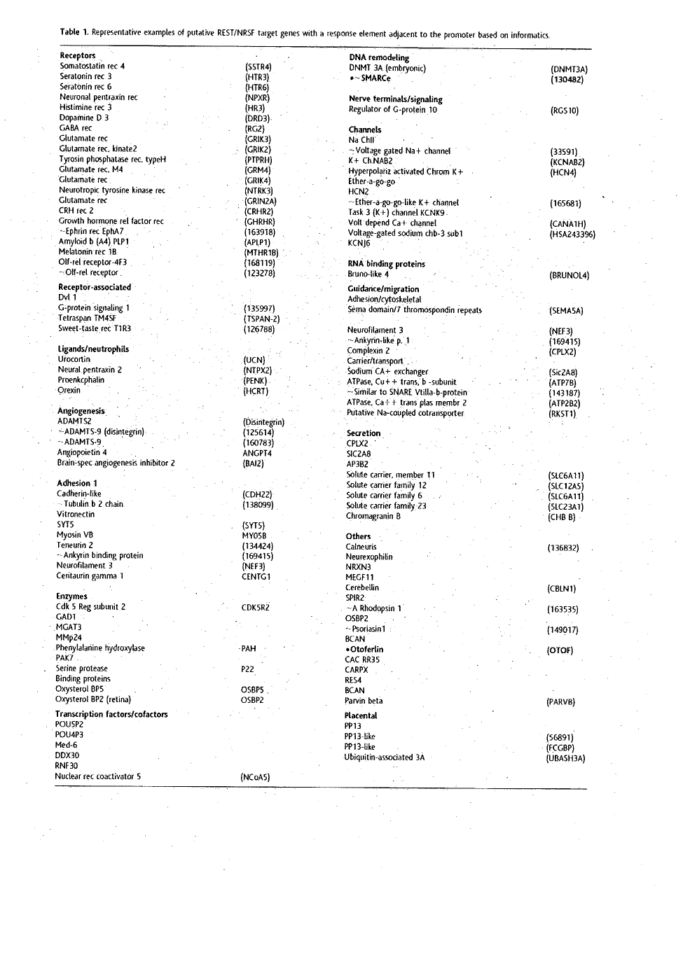

It can be concluded in the present invention based upon the data described that the zinc-finger factor REST/NRSF can mediate both active repression via recruitment of specific HDACs and gene silencing by recruitment of CoREST complexes (You et al., 2001, Proc. Natl. Acad. Sci. U.S.A., 98: 1454-59; Humphrey et al., 2001, J. Biol. Chem., 276: 6817-22; Hakimi et al., 2002, Proc. Natl. Acad. Sci. U.S.A., 99: 7420-24) to specific promoters in a cell type- and promoter-specific DNA methylation manner. Similar events occur in vivo, with CoREST, MeCP2, and Km9H3 markers of silencing proving to be present on the NaChII promoter in adult murine liver and heart (FIG. 5A). With the use of MEME and SP-STAR motif-finding algorithms (Mieda et al., 1997, J. Biol. Chem., 272: 5854-59), two motifs were located that are present preferentially, within 250 bp of an experimentally confirmed RE1I/NRSE consensus site that may be related to the known RE1/NRSE consensus site. The full importance of these motifs will need to be genetically studied, but one (RE2) is capable of both transcriptional repression and REST/NRSF binding. Thus, analogous to nucleation of gene silencing at specific sequences [polycomb group response elements (PRE's)] Schoenherr et al., 1995, Science, 267: 1360-64), it can be suggested that the RE1/NRSE element, perhaps in concert with related sites, might nucleate silencing of specific chromosomal regions. While the presently described invention has identified two motifs that were located preferentially within 250 bp of an RE1/NRSE consensus site, it is in no way limited to these two motifs and there may be other as yet undiscovered or uncharacterized motifs that play roles in recruiting biochemical complexes or driving nucleation required for chromosomal interval silencing.

Recruitment of the co-repressor CoREST to REST/NRSE gene targets appears to act as a molecular beacon for the silencing machinery, including MeCP2, SUV39H1, and HP1, to propagate and maintain a methyl CpG-dependent silent state across specific chromosomal intervals, including genes that do not contain REST/NRSF-binding sites (FIG. 5B). This model is consistent with observations that DNA methylation by itself is not sufficient for silencing (Jones et al., 2002, Nature Rev. Genet., 3: 415-420). MeCP2 appears to be a critical component of these events in Rat-1 cells, but other factors and molecular beacons may operate in cell types where MeCP2 is not expressed and are therefore covered by the present invention. The recruitment of SUV39H1, in part via interactions with MeCP2 complexes, apparently leads to HP1 recruitment and chromatin condensation in cultured cells and in vivo (FIG. 5B). The presence of Km9 but not Km4 histone H3 across the rCh3 q22-34 region is consistent with CoREST-mediated recruitment of silencing machinery and the proposed epigenetic program (Heard et al., 2001, Cell, 107: 727-32; Rea et al., 2000, Nature, 406: 593-98; Noma et al., 2001, Science, 293: 1150-55; Nakayama et al., 2001, Science, 292: 10-15; Wood et al., 1996, J. Biol. Chem., 271: 14221-26).

These observations further suggest that other factors analogous to REST are likely to mediate the silencing of distinct chromosomal regions that regulate other biological programs, some via recruitment of CoREST complexes. Conversely, the expression of REST/NRSF early in brain development and the potential silencing of genes such as SMARCE suggest that REST/NRSF may also control important roles in early embryonic gene silencing.

While the presently described invention relates to the description of REST/NRSF function as it pertains to both short-term active repression and long-term gene silencing it is in no way limited to this particular transcription factor. Other transcription factors may act in similar biochemical complexes and through similar molecular events to drive the repression and or silencing of target gene expression. Other transcription factors of prokaryotic, eukaryotic and viral origin contemplated and therefore covered by the presently described invention include, but are not limited to A2, AAF, abaA abd-A, Abd-B, ABF1, ABF-2, ABI4, Ac, ACE2, ACF, ADA2, ADA3, ADA-NF1, Adf-1, Adf-2a, Adf-2b, ADR1, AEF-1, AF-1, AF-2, AFLR, AFP1, AFX-1, AG, AG1, AG2, AG3, AGIE-BP1, AGL11, AGL12, AGL13, AGL14, AGL15-1, AGL15-2, AGL17, AGL2, AGL3, AGL4, AGL6, AGL8, AGL9, AhR, AIC3, AIC2, AIC3, AIC4, AIC5, AID2, AIIN3, ALF1B, ALL-1, alpha-1, alpha2uNF1, alpha2uNF2, alph2uNF3, alpha-CP1, alpha-CP2a, alpha-CP2b, alpha-factor, alphaH0, alphaH2, alphaH3, alpha-IRP, alpha-PAL, alpha2uNF1, alpha2uNF3, alphaA-CRYBP1, alphaH2-alphaH3, alphaMHCBF1, Alx-3, Alx-4, ALY, AMDA, AmdR, aMEF-2, AML1, AML1a, AML1b, AML1c, AML1DeltaN, AML2, AML3, AMT1, AMY-1L, A-Myb, AN2, AnCF, ANF, ANF-2, ANR1, Antp, AP-1, AP-2, AP-2alphaisoform2, AP-2alphaisoform3, AP-2alphaisoform4, AP-3, AP3-1, AP3-2, AP4, AP-5, APC, APETALA1, APETALA3, AR, ARA, AREA, AREB6, ARG RI, ARG RII, armadillo, Arnt, ARP-1, ARP7, ARP9, ARR1, AS-C T3, AS321, ASF-1, ASH-1, ASH-3b, ASP, AT-13P2, ATBF1-A, ATBP, AT-BP1, AT-BP2, ATF, ATF-1, ATF-3, ATF-3deltaZIP, ATF-adelta, ATF-like, Athb-1, Athb-2, Ato, Axial, AZF1, B factor, B″, BAF1, B-TFIID, band I factor, BAP, Barx-1, BAS, BBF1, BBF2a, BBF3, BBFa, Bcd, BCFI, BcL-3, BCL-6, BD73, BDF1, beta-1, BETA1, BETA2, beta-catenin, beta-factor, BF-1, BF-2, BGP1, Bin1, Blimp-1, BmFTZ-F1, B-Myb, B-Myc, BP1, BP2, B-Peru, BR-C Z1, BR-C Z2, BR-C Z4, Brachyury, BRF1, Br1A, Brn-3a, Brn-4, Brn-5, BUF1, BUF2, BAF1, BAS1, BCFII, beta-factor, BETA3, BLyF, BP2, BR-C Z3, brachyuray, brahma, BRF1, Brn1, Brn2, Brn-3a, Brn-3b, Brn-4, Brn-5, Bro, Btd, BTEB, BTEB2, BUF, BUF1, BUF2, BUR6, byr3, BZIP910, BZIP911, c-ab1, c-Ets-1, c-Ets-2, c-Fos, c-Jun, c-Maf, c-myb, c-Myc, c-Qin, c-Rel, C/EBP, C/EBPalpha, C/EBPbeta, C/EBPdelta, C/EBPepsilon, C/EBPgamma, C1, CAC-binding protein, CACCC-binding factor, Cactus, Cad, CAD1, CAF17, CAL, CAP, CAR2, CArG box-binding protein, CAT8, CAUP, CBF1, CBF2, CBF3, CBF4, CBF5, CBF-A, CBF-B, CBF-C, CBP, CBTF, CCAAT-binding factor, CCBF, CCF, CCG1, CCK-1a, CCK-1b, CCR4, CD28RC, CDC10, Cdc68, CDF, cdk2, CDP, CDP2, Cdx-1, Cdx-2, Cdx-3, Cdx-4, CEBF, CEF1, ceh-1, ceh-10, ceh-12, ceh-13, ceh-14, ceh-16, CEH-18 and (all ceh related factors), CeMyoD, c-Ets-1, C-Ets-1A, c-Ets-1B, CF1, Cf1a, CF2-I, CF2-II, CF2-III, CFF, CG-1, CHA4, CHOP-10, Chox-2.7, Chx10, CIN5, CIIIB1, c-Jun, CKB3, Clox, c-Maf, CMB1, CMB2, c-Myb, c-Myc, CNBP, Cnc, CoMP1, core-binding factor, CoS, COUP, COUP-TF, CP1, CP1A, CP1B, CP1C, CP2, CPBP, CPC1, CPE binding protein CPRF-1, CPRF-2, CPRF-3, CPM10, CPM5, CPM7, CPPI, CPRF-1, CPRF-2, CPRF-3, CPRF-4a, CPRF-4b, all CREB related factors, CRE-BP1, CRE-BP2, CRE-BP3, CRE-BPa, CreA, CREB, CREB-2, CREBomega, CREMalpha, CREMbeta, CREMdelta, CREMepsilon, CREMgamma, CREMtaualpha, CRF, all CRM related factors, Croc, Crx, CRZ1, CSBP-1, CtBP, CTCF, CTF, CUM1, CUM10, CUP2, CUP9, CUS1, Cut, Cux, CWH-1, CWH-2, CWH-3, Cx, cyclin A, cyclin T, cyclin T1, cyclin T2, cyclin T2a, cyclin T2b, CYS3, D-MEF2, Da, all DAL related factors, DAP, DAPI, DAT1, DAX1, DB1, DBF-A, DBF4, DBP, DBSF, dCREB, DDB, DDB-1, DDB-2, dDP, dE2F, DEAP3, DEF, DEFH2, Delilah, delta factor, deltaCREB, deltaE1, deltaEF1, deltaMax, DENF, DENF1, DENF2, DENF3, DEP, DEP2, DEP3, DEP4, DERmo-1, DF-1, DF-2, DF-3, Dfd, dFRA, DHR3, DHR38, DHR78, DHR96, dioxin receptor, dJRA, D1, DII, all D1x related factors, DM-SSRP1, DMLP1, Dof3, DP-1, DP-2, Dpn, Dr1, all DREB related factors, DRF1, DRF2, DRTF, DSC1, DSIF, DSP1, DST1, DSXF, DSXM, DTF, E, E1A, E2, E2BP, E2F, E2F-BF, E2F-I, E4, E47, E4BP4, E4F, E4TF2, E7, E74, E75, EAP1, EAP2L, EAP2S, EAR2, EBF, EBF1, EBNA, EBP, EBP40, EC, EC5, ECF, ECF2, ECF3, ECH, ECM22, EcR, eE-TF, EF-1A, EF-C, EF1, EFgamma, EGM1, EGM2, EGM3, Egr, EGR2, EGR3, eH-TF, EIIa, EivF, EKLF, Elf-1, Elg, Elk-1, ELP, Elt-2, EmBP-1, embryo DNA binding protein, Emc, EMF, EMF2, EMF3, EMF4, Ems, Emx, Emx-1, Emx-2, En, ENH-binding protein, ENKTF-1, epsilonF1, ER, Erbeta, EREBP-1, EREBP-2, EREBP-3, EREBP-4, ERF1, Erg, Esc, Esc1, esg, Esx-1a, Esx-1b, ETF, ETL, Eve, Evi, Evx, Exd, Ey, en-1, en-2, f(alpha-f(epsilon), F27E5.2, F2F, FACB, F-ACT1, factor 1, factor 2, factor 3, factor B1, factor B2, factor delta, factor I, FAR, Fbf1, FBF-A1, FBP, FBP1, FBP11, FBP2, FBP6, FBP7, f-EBP, FHL1, FIM, FKBP59, Fkh, FKH1, Fkh-1, FKH2, Fkh-2, Fkh-3, Fkh-4, Fkh-5, Fkh-6, FKHR, FKHRL1, FKHRL1P1, FKHRL1P2, FKHRP1, FlbD, FLC, FLF, Flh, Fli-1, FLO, FLO8, FLV-1, FOG, FosB, FosB/SF, Fra-1, Fra-2, Freac-1, Freac-10, Freac-2, Freac-3, Freac-4, Freac-5, Freac-6, Freac-7, Freac-8, Freac-9, FRG Y1, FRG Y2, FTF, FTS, Ftz, FTZ-F1, FTZ-F1beta, FZF1G factor, G factor, G/HBF-1, G10BP, G6 factor, GA-BF, GABP, GABP-alpha, GABP-beta1, GABP-beta2, GAF, GAF1, GAF2, GAG2, GAL11, GAL4, GAL80, GammaCAAT, gammaCAC1, gammaCAC2, gamma-factor, gammaOBP, GAMYB, GAT1, GAT2, GAT3, GAT4, GATA-1, GATA-1A, GATA-1B, GATA-2, GATA-3, GATA-4, GATA-5, GATA-5A, GATA-, GATA-6, GATA-6A, GATA-6B, GBF, GBF1, GBF12, GBF1A, GBF1B, GBF2, GBF2A, GBF2B, GBF3, GBF4, GBF9, GBP, GC1, GC2, GC3, GCF, GCM, GCMa, GCMb, GCN4, GCN5, GCNF, GCR1, GCR2, GE1, GEBF-I, GF1, GFI, Gfi-1, GFII, GHF3, GHF-5, GHF-7, GIS1, GKLF, GL1, Gl15, Gl2, Glass, GLI, GLI3, GLN3, GLO, GM-PBP-1, GP, GR, GR alpha, GR beta, GRF-1, Grg-4, Grg-5, GRIP1, Groucho, Gsb, GSBF1, Gsbn, Gsc, Gsc A, Gsc B, Gt, GT-1, GT-2, GT-IC, GT-IIA, GT-IIBalpha, GT-IIBbeta, GTS1, Gtx, GZF3, H16, H1TF1, H1TF2, H2B abp 1, H2RIIBP, H4TF-1, H4TF-2, HAC1, HAL9, HALF-1, HAP1, HAP2, HAP3, HAP4, HAP5, Hb, HB9, HBLF, HBP-1, HBP-1a, HBP-1a(1), HBP-1a(c14), HBP-1b, HBP-1b(c1), HCM1, HDaxx, heat-induced factor, HEB, HEB1-p67, HEB1-p94, HEF-1B, HEF-1T, HEF-4C, HEN1, HEN2, HeRunt-1, HES-1, HES-2, HES-3, HES-5, Hesx1, Hex, HFH-1, HFH-11A, HFH-11B, HFH-2, HFH-3, HFH-4, HFH-5, HFH-6, HFH-7, HFH-8, HIF-1, HIF-1alpha, HIF-1beta, HiNF-A, HiNF-B, HiNF-C, HiNF-D, HiNF-D3, HiNF-E, HiNF-M, HiNF-P, HIP1, HIR1, HIR2, HIR3, HIRA, HIV-EP2, H1f, H1f-alpha, H1f-beta, HLX, H1x, HMBP, HMG I, HMG I(Y), HMG Y, HMGI-C, HMS1, HMS2, HNF-1, HNF-1A, HNF-1B, HNF-1C, HNF-3, HNF3(-like), HNF-3alpha, HNF-3B, HNF-3beta, HNF-3gamma, HNF-4, HNF-4(D), HNF-4alpha1, HNF-4alpha2, HNF-4alpha3, HNF-4alpha4, HNF-4alpha7, HNF-4beta, HNF-4gamma, HNF-6, HNF-6alpha, HNF-6beta, hnRNP K, Hox11, HOXA1, HOXA10, HOXA10 PL2, HOXA11, HOXA13, HOXA2, HOXA3, HOXA4, HOXA5, HOXA6, HOXA7, HOXA9, HOXB1, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC6 (PRI), HOXC6 (PRII), HOXC8, HOXC9, HOXD1, HOXD10, HOXD11, HOXD12, HOXD13, HOXD3, HOXD4, HOXD8, HOXD9, HP1 site factor, Hp55, Hp65, HrpF, HSE-binding protein, HSF, HSF1, HSF2, HSF24, HSF30, HSF8, hsp56, Hsp90, HST, HSTF, HY5, IBF, IBP-1, IBR, ICER, ICER-I, ICER-Igamma, ICER-II, ICER-Iigamma, ICP4, ICSBP, Id1, Id1.25, Id1H′, Id2, Id3, Id3/Heir-1, Id4, IDS1, IE1, IEBP1, IEFga, IF1, IF2, IFH1, IFNEX, IgPE-1, IgPE-2, IgPE-3, Ik-1, Ik-2, Ik-3, Ik-4, Ik-5, Ik-6, Ik-7, Ik-8, IkappaB, IkappaB-alpha, IkappaB-beta, IkappaB-gamma, IkappaB-gamma1, IkappaB-gamma2, IkappaBR, IKI3, ILF, ILRF-A, IME1, IME4, INO2, INO4, INSAF, IPF1, I-POU, IRBP, IRE-ABP, IREBF-1, IRF-1, IRF-2, IRF-3, ir1B-2a, Irx-3, ISGF-1, ISGF-3, ISGF-3alpha, ISGF-3gamma, Isl-1, ISRF, ISRFI, ITF, ITF-1, ITF-2, IUF-1, Ixr1, JRF, Jun-D, JunB, JunD, K06B9.5, K07C11.1, kappaY factor, KAR4, KBF2, kBF-A, KBP-1, KCS1, KER1, -1, Kid-1, Kin17, KN1, Kni, Knox3, KNRL, Kox1, Kr, Kreisler, KRF-1, Krox-20, Krox-24, Ku autoantigen, KUP, Lab, LAC9, LBP, LBP-1, LBP-1a, Lc, LCR-F1, LD, Ldb1, LEF-1, LEF-1B, LEF-1S, LEU3, LF-A1, LF-A2, LF-B2, LF-C, LFY, LG2, LH-2, Lhx-3, Lhx-3a, Lhx-3b, Lhx-4, LHY, Lim-1, Lim-3, lin-1, lin-11, lin-14A, lin-14B1, lin-14B2, lin-29A, lin-29B, lin-31, lin-32, lin-39, LIP15, LIP19, LIT-1, LKLF, Lmo1, Lmo2, Lmx-1, L-Myc1, L-Myc-1, L-Myc-1(long form), L-Myc-1(short form), L-Myc-2, LR1, LSF, LSIRF-2, LUN, Lva, LVb-binding factor, LVc, LXRalpha, LyF-1, Lyl-1, LYS14, Lz, M factor, M-Twist, M1, m3, Mab-18, MAC1, Mad, MAF, MafB, MafF, MafG, MafK, Ma163, MAPF1, MAPF2, MASH-1, MASH-2, mat-Mc, mat-Pc, MATa1, MATalpha1, MATalpha2, MATH-1, MATH-2, Max1, M factor, M1, m3, Mab-18 (284 AA), Mab-18 (296 AA), mab-5, MAC1, Mad1, Mad3, Mad4, MADS1, MADS11, MADS16, MADS2, MADS24, MADS3, MADS4, MADS45, MADS5, MADS6, MADS7, MADS8, MADS9, MAF, MafB, MafF, MafG, MafK, MAL13, MAL23, MAL33, MAL63, MAPF1, MAPF2, MASH-1, MASH-2, Mat1-Mc, MATa1, MATalpha1, MATalpha2, MATH-1, MATH-2, mat-Pc, Max, Max1, Max2, MAZ, MAZi, MB67, MBF1, MBF-1, MBF2, MBF3, MBF-I, MBP1, MBP-1 (1), MBP-1 (2), MBP-2, MCBF, MCM1, MCM1+MATalpha1, MDBP, MDBP-2, MDS3, mec-3, MECA, MED11, MED2, MED4, MED6, MED7, MED8, mediating factor, MEF1, MEF-2, MEF-2B, MEF-2B-1, MEF-2B-2, MEF-2B-3, MEF-2B-4, MEF-2C, MEF-2C (433 AA form), MEF-2C (465 AA form), MEF-2C (473 AA form), MEF-2C/delta32 (441 AA form), MEF-2D, MEF-2D (506 AA form), MEF-2D (514 AA form), MEF-2D00, MEF-2D0B, MEF-2DA [symbol] 0, MEF-2DA [symbol] B, MEF-2DA0, MEF-2DAB, Meis-1, Meis-1-1, Meis-1-2, Meis-1-3, Meis-1-4, Meis-1a, Meis-1b, Meis-2a, Meis-2b, Meis-2c, Meis-2d, Meis-3, Meso1, MET18, MET28, MET31, MET32, MET4, Mf2, MF3, MFH-1, Mfh-1, MGA1, Mhox, MHR1, Mi, MIBP1, MIF-1, MIG1, MIG2, Mix.1, Mix.2, Mix.3, Mix.4, Mixer, MIXTA, Miz-1, MKR2, MLP, MM-1, MNB1a, MNB1b, MNF1, MNR2, MOK-2, MOP3, MOT1, MOT3, MP4, MPBF, MR, MRF4, MRR, Msh, MSN1, MSN2, MSN4, Msx-1, Msx-2, MTB-Zf, MTF1, MTF-1, MTH1, Mt11, mtTF1, M-Twist, muEBP-B, muEBP-C2, MUF1, MUF2, Mxi1, MYB A, MYB.PH1, MYB.PH2, MYB.PH3, MYB1, Myb-1, all Myb related proteins, MYB-P1, MYBST1, myc-CF1, myc-PRF, MYC-RP, Myef-2, Myf-3, Myf-4, Myf-5, Myf-6, Myn, MyoD, Myogenin, MZF-1, Nab1, Nau, NBF, NC1, NCB2, NDT80, NELF, NeP1, NER1, Net, NeuroD, NF III-a, NF III-c, NF III-e, NF-1, NF-1/L, NF-1/Red1, NF-1A, NF-1A1, NF-1A1.1, NF-1A2, NF-1A3, NF-1A4, NF-1A5, NF-1B, NF-1B1, NF-1B2, NF-1B3, NF-1B4, NF-1C1, NF-1C2, NF-1C4, NF-1X, NF-1X1, NF-1X2, NF-1X3, NF2d9, NF-4FA, NF-4FB, NF-4FC, NF-A, NF-A3, NF-AB, NFalpha1, NFalpha2, NFalpha3, NFalpha4, NF-AT, NFAT-1, NF-AT3, NF-Atc, NF-ATc3, NF-Atp, NF-Atx, NF-BA1, NfbetaA, NF-CLE0a, NF-CLE0b, NF-D, NFdeltaE3A, NFdeltaE3B, NFdeltaE3C, NFdeltaE4A, NFdeltaE4B, NFdeltaE4C, Nfe, NF-E, NF-E1b, NF-E2, NF-E2 p45, NF-E3, NF-E4, NFE-6, NF-EM5, NF-Gma, NF-GMb, NF-H1, NF-H2, NF-H3, NFH3-1, NFH3-2, NFH3-3, NFH3-4, NF-IL-2A, NF-IL-2B, NF-InsE1, NF-InsE2, NF-InsE3, NF-jun, NF-kappaB, NF-kappaB(-like), NF-kappaB1, NF-kappaB1 precursor, NF-kappaB2, NF-kappaB2 (p49), NF-kappaB2 precursor, NF-kappaE1, NF-kappaE2, NF-kappaE3, NF-lambda2, NF-MHCIIA, NF-MHCIIB, NF-muE1, NF-muE2, NF-muE3, NF-muNR, NF-ODC1, NF-S, NF-TNF, NF-U1, NF-W1, NF-W2, NF-X, NF-X1, NF-X2NF-X3, NF-Xc, NF-Y, NF-Y′, NF-YA, NF-YB, NF-YC, NF-Zc, NF-Zz, NGFI-B, NGFI-C, NHP-1, NHP-2NHP3, NHP4, NHR1, NIP, NIRA, NIT2, NIT4, Nkx-2.1, Nkx-2.2, Nkx-2.5, NLS1, NMH7, NMHC5, Nmi, N-Myc, N-Myc1, N-Myc2, nob-1A, nob-1B, N-Oct-2alpha, N-Oct-2beta, N-Oct-3, N-Oct-4, N-Oct-5a, N-Oct-5b, NOR1, NOT, NOT1, NOT2, NOT3, NOT5, NP-III, NP-IV, NP-TCII, NP-Va, NPX1, NRD I, Nrf1, NRF-1, Nrf2, NRF-2NRF-2beta1, NRF-2gamma1, NRFA, NRG1, NRG2, NRL, NS-1, NSDD, NTF, NTF1, NUC-1, Nur77, NUT1, NUT2, OBF, OBF-1, OBF3.1, OBF3.2, OBF4, OBF5, OBP, OBP1, OC-2, OCA-B, OCSBF-1, OCSTF, Oct-1, Oct-10, Oct-11, Oct-1A, Oct-1B, Oct-1C, Oct-2, Oct-2.1, Oct-2.3, Oct-2.4, Oct-2.6, Oct-2.7, Oct-2.8, Oct-2B, Oct-2C, Oct-4, Oct-4A, Oct-4B, Oct-5, Oct-6, Oct-7, Oct-8, Oct-9, Octa-factor, octamer-binding factor, oct-B2, oct-B3, Oct-R, Odd, ODR7, OG-12, OG-2, OG-9, OHP1, OHP2, Olf-1, OM1, ONR1, Opaque-2, OPM1, OSBZ8, Otd, Otx1, Otx2, Otx4, Ovo, OZF, P (long form), P (short form), P1, p107, p130, p28 modulator, p300, p38erg, p40x, p45, p49erg, p53as, p55, p55erg, p58, p65delta, p67, PAB1, PacC, PAF1, pag-3, PAGL1, pal-1, Pap1+, par-2, Paraxis, PARP, Pax-1, Pax-1/9, Pax-1/9 (AmphiPax-1), Pax-1/9-I, Pax-1/9-II, Pax-1/9-III, Pax-1/9-IV, Pax-1/9-V, Pax-1/9-VI, Pax-2, Pax-2.1, Pax-2.2, Pax-2/5/8, Pax-2a, Pax-2b, Pax-3, Pax-3A, Pax-3B, Pax-4, Pax-4a, Pax-4b, Pax-4c, Pax-4d, Pax-5, Pax-6, Pax-6 (Pax-QNR), Pax-6/Pd-5a, Pax-6 12.1, Pax-6 12.2, Pax-6 4.1, Pax-6 4.2, Pax-6 J2, Pax-7, Pax-8, Pax-8a, Pax-8b, Pax-8c, Pax-8d, Pax-8c, Pax-8f, Pax-8g, Pax-9, Pax-A, Pax-B, Pb, PBF, PBP, Pbx-1a, Pbx-1b, Pc, PC2, PC4, PC4 p9, PC5, Pcr1, PCRE1, PCT1, PDM-1, PDM-2, PDR1, PDR3, Pdx-1, PEA1, PEA2, PEA3, PEB1, PEBP2, PEBP2alpha, PEBP2alphaA/Osf2, PEBP2alphaA/til-1, PEBP2alphaA/til-1 (Y), PEBP2alphaA/til-1(U), PEBP2alphaA1, PEBP2alphaA2, PEBP2alphaB1, PEBP2alphaB2, PEBP2beta, PEBP2beta1, PEBP2beta2, PEBP2beta3, PEBP5, Pep-1, PERIANTHIA, pes-1apes-1b, PF1, PF3, PGA4, PGD1, pha-4, PHAN, PHD1, phiAP3, PHO2, PHO4, PHO80, Phox-2, php-3, PI, PI1, PI2, pie-1, PIHbox9, PIP2, Pit-1, Pit-1a, Pit-1b, Pit-1c, Pitx-3, PLE, PLE/DEFH200, PLE/DEFH49, PLE/DEFH72, PLE/SQUA, PLZF, PNPI2, PO-B, pointedP1, pointedP2, Pontin52, pop-1POP2, POTM1-1, pou[c], Pou2, pox neuro, PP1, PP2, PPAR, PPARalpha, PPARbeta, PPARgamma, PPR1, PPUR, PPYR, PR, PR A, PRb, Prd, PRDI-BF1, PRDI-BFc, PREB, Prop-1, protein a, protein b, protein c, protein d, PRP, PSE1, Psx-1, Psx-2, P-TEFb, PTF, PTF1, PTF1-alpha, PTF1-beta, PTFalpha, PTFbeta, PTFdelta, PTFgamma, Ptx-1, Ptx-2, Ptx-2B, Pu box binding factor, Pu box binding factor (BJA-B), PU.1, Pu.1, PUB1, PuF, PUF-I, Pur factor, Pur-1, PUT3, P-wr, PX, PZF1, qa-1F, QBP, QUT1, R, R1, R2, RAD1, Rad-1, RAD18, RAD2, RAF, RAP1, RAP2.5, RAR, RAR-alpha, RAR-alpha1, RAR-alpha2, RAR-beta, RAR-beta1, RAR-beta2, RAR-beta3, RAR-beta4, RAR-gamma, RAR-gamma1, RAR-gamma2, RAV1, RAV2, Rax, Rb, RBP60, RBP-Jkappa, Rc, RC1, RC2, RCS1, REB, REB1, Reb1p, Re1A, Re1B, repressor of CAR1 expression, REV-ErbAalpha, REX-1, RF1, RF2a, RFX, RFX1, RFX2, RFX3, RFX5, RF-Y, RGM1, RGR1, RGT1, RIC1, RIM1, RIP14, RITA-1, RLM1, RME1, RMS1, Ro, Roaz, ROM1, ROM2, RORalpha1, RORalpha2, RORalpha3, RORbeta, RORgamma, Rox, Rox1, ROX3, RPF1, RPGalpha, RPH1, RREB-1, RRF1, RRF2, RRF3, RRN10, RRN11, RRN3, RRN5, RRN6, RRN7, RRN9, RS2, RSC4, RSRFC4, RSRFC9, RSV-EF-II, RTF1, RTG1, RTG2, RTG3, Runt, RVF, Rx, Rx1, Rx2, Rx3, RXR-alpha, RXR-beta, RXR-beta1, RXR-beta2, RXR-gamma, S8, SAP1, SAP-1a, SAP-1b, SBF, SBF-1, Sc, SCBPalpha, SCBPbeta, SCBPgamma, SCD1/BP, SCM-inducible factor, Scr, S-CREM, S-CREMbeta, Sd, Sdc-1, SDS3, SEF1, SEF-1 (1), SEF-1 (2), SEF3, SEF4, SEM-4, SET1, SET2, SF1, SF-1, SF-2, SF-3, SF-A, SFL1, SGC1, SGF-1, SGF-2, SGF-3, SGF-4, Shn, SHP, SHP1, SHP2, SIF, SIG1, SIII, SIII-p110, SIII-p15, SIII-p18, Sim1, Sim2, Six-1, Six-2, Six-3, Six-3alpha, Six-3beta, Six-4, Six-4A, Six-4B, Six-4C, Six-5, Six-6, Skn-1, SKN7, SKO1, SLM1, SLM2, SLM3, SLM4, SLM5, S1p1, s1p2, S-Myc, Sn, SN (sienna), Sna, SNF5, SNF6, SNP1, So, SOX-11, SOX-12, Sox-13, SOX-15, Sox-18, Sox-2, Sox-4, Sox-5, SOX-6, SOX-9, Sox-LZ, Sp1, Sp2, Sp3, Sp4, SPA, spE2F, Sph factor, Spi-B, SpOtx, Sprm-1, SpRunt-1, SQUA, SRB10, SRB11, SRB2, SRB4, SRB5, SRB6, SRB7, SRB8, SRB9, SRD1, SRE BP, SREBP-1, SREBP-1a, SREBP-1b, SREBP-1c, SREBP-2, SREP, SRE-ZBP, SRF, SRY, Sry h-1, Sry-beta, Sry-delta, ssDBP-1, ssDBP-2, SSRP1, Staf, Staf-50, STAT, STAT1, STAT1alpha, STAT1beta, STAT2, STAT3, STAT4, STAT5, STAT5A, STAT5B, STAT6, STC, STD1, Ste11, STE12, STE4, STF1, STF2, STKA, STM, STP1, Stra13, StuAp, su(f), Su(H), su(Hw), SUM-1, SUP, SVP, SVP46, SWI/SNF complex, SWI1, SWI2, SWI3, SWI4, SWI5, SWI6, SWP, T-Ag, t-Pou2, T3R, T3R-alpha, T3R-alpha1, T3R-alpha2, T3R-beta, T3R-beta1, T3R-beta2, TAB, T-Ag, TAG1, Tal-1, Tal-1beta, Tal-2, TAR factorTat, Tax, TCF, TCF-1TCF-1A, TCF-1B, TCF-1C, TCF-1D, TCF-1E, -1F, TCF-1G, TCF-2, TCF-2alpha, TCF-3, TCF-3B, TCF-3C, TCF-3D, TCF-4, TCF-4(K), TCF-4B, TCF-4E, TCF-A, TCF-B, TCFbeta1, TDEF, TEA1, TEC1, TEF, TEF 1, TEF-1, TEF2, TEF-2, Te1, TF68, TFE3, TFE3-L, TFE3-S, TFEB, TFEC, TFIIA, TFIIA (13.5 kDa subunit), Tf-LF1, Tf-LF2, TF-Vbeta, TGA, TGA1, TGA1a, TGA2, TGA3, TGA6, TgF1, TGGCA-binding protein, TGT3, Th1, THM1, THM18, THM27, THRA1, TIF1, TIF2, TIN-1, TINY, TIP, tI-POU, TLE1, Tll, Tlx, TM3, TM4, TM5, TM6, TM8, TMF, t-Pou2, TR2, TR2-11, TR2-9, TR3, TR4, Tra-1 (long form), Tra-1 (short form), TRAP, TREB-1, TREB-2, TREB-3, TREF1, TREF2, TRF, TRF (2), Trident, TSAP, TSF3, Tsh, TTF-1, TTF-2, TTG1, Ttk 69K, Ttk 88K, TTP, Ttx, ttx-3, TUBF, Twi, TxREF, TyBF, UAY, UBF, UBF1, UBF2, UBP-1, Ubx, UCRB, UCRF-L, UEF-1, UEF-2, UEF-3, UEF-4, UF1-H3beta, UFA, UFB, UFO, UGA3, UHF-1, UME6, unc-30, unc-37, unc-4, Unc-86, URF, URSF, URTF, USF, USF2, vab-3, vab-7, vaccinia virus DNA-binding protein, Vav, Vax-1, Vax-2, VBP, VDR, v-ErbA, VETF, v-Ets, v-Fos, vHNF-1, vHNF-1A, vHNF-1B, vHNF-1C, VITF, v-Jun, v-Maf, Vmw65, v-Myb, v-Myb/v-Ets, V-Myc, v-Myc, Vp1, Vpr, v-Qin, v-Re1, VSF-1, WC1, WC2, Whn, WT1, WT1I, WZF1, X-box binding protein, X-Twist, X2BP, xam1, X-box binding protein, XBP-1, XBP-2, XBP-3, XF1, XF2, XFD-1, XFD-2, XFD-3, XFG20, XGRAF, Xiro1, Xiro2, Xiro3, xMEF-2, XPF-1, XrpF1, XW, XX, yan, YB-1, YB-3, Ybx-3, YEB3, YEBP, Yi, YNG2, YPF1, YY1, ZAP, ZEB, ZEM1, ZEM2/3, Zen-1, Zen-2, Zeste, ZF1, ZF2, ZF5, Zfh-1, Zfh-2, Zfp-35, ZID, ZIP-1A, ZIP-2A, ZIP-2B, ZM1, ZM38, Zmhoxla, Zn-15, ZNF174, ZPT2-1, ZPT2-2, ZPT2-3, ZPT2-4, Zta. In addition, any factors which retain the ability to repress gene transcription either through short-term active repression or long-term gene silencing, and are as of yet previously undiscovered or as uncharacterized are covered by the present invention.

### 6.0 EXAMPLES

6.1 REST/NRSF Mediation of HDAC-dependent and DNA methylation-dependent Repression of Neuronal-specific Genes in Rat-1 Fibroblasts

The present invention outlines two mechanisms by which REST/NRSF may mediate the downregulation of target gene activity. REST/NRSF has the capacity to drive short-term active repression via the recruitment of HDAC activity as well as long-term gene silencing through the recruitment of a biochemical complex containing histone methyltransferase activity. FIG. 1 outlines the presence of different biochemical components for each mechanism on the target genes NaChII and SCG10. (A) outlines ChIP results in Rat-1 fibroblasts, using PCR primers specific for the REST/NRSF-containing regions of NaChII and IgG's specific to CoREST, REST/NRSF, and N—CoR. The data indicate that CoREST and REST/NRSF, but not the co-repressor N—CoR are present on the NaChII promoter. (B) characterizes SCG10 and NaChII transcripts with transient expression of RESTDBD or RESTWT in Rat-1 cells as detected by reverse transcriptase PCR (RT-PCR). Both genes were derepressed in the presence of the dominant negative DNA binding domain alone of REST/NRSF but not wild-type REST. PC12 cells and constitutively expressed glyceraldehydes-3-phosphate dehydrogenase (GAPDH) were used as controls. (C) demonstrates that NaChII but not SCG10 gene repression is reversed by transient expression of a dominant negative CoREST RID. (D) shows that ectopic activation of the SCG10 gene is observed in TSA-treated (300 nM) Rat-1 fibroblasts. No change in the repression of the NaChII gene was observed in the presence of TSA. (E) depicts mapping of the methylation status of the NaChII gene by bisulfite-modification sequencing in Rat-1 cells, in the promoter (upper) and further 3′ region within the NaChII gene (lower). Methylated CpG pairs are shown as open boxes, with unmethylated C converted to T (underlined) upon bisulfite treatment. (F) reveals a change in DNA methylation status upon treatment with the demethylating agent 5AzaC and (G) reveals a restoration of NaChII gene expression after 5AzaC treatment in Rat-1 cells. Rat-1 cells were treated with 10 uM of 5AzaC in the absence (−) or presence (+) of 300 nM of TSA. Total RNA was isolated for the detection of NaChII or SCG10 mRNA by RT-PCR.

6.2 MeCP2 represses transcription from the endogenous methylated NaChII gene in Rat-1 fibroblasts.

MeCP2 has been shown to bind specifically to methylated CpG dinucleotdies and repress transcription of gene activity in an HDAC-independent fashion (Tate et al., 1993, Curr. Opin. Genet. Dev., 3: 226-31; Siegfried et al., 1999, Nature Genet., 22: 203-08; Lewis et al., 1992, Cell, 69: 905-09; Lorincz et al., 2001, Mol. Cell Biol., 21: 7913-18; Amir et al., 1999, Nature Genet., 23: 185-90; Chen et al., 2001, Nature Genet., 27:327-32; Bell et al., 2000, Nature, 405: 482-87). Mutations in MeCP2 are linked to derepression of target gene activity and result in the progressive neurodegenerative disorder Rett syndrome (Amir et al., 1999, Nature Genet., 23: 185-90). Because MeCP2 is expressed highly in Rat-1 cells and has been functionally linked to gene silencing its putative role in the silencing of the NaChII gene was investigated. FIG. 2 depicts the presence of MeCP2 on the endogenous methylated NaChII gene and its requirement for silencing of this locus. (A) demonstrates that MeCP2 interacts with the endogenous promoter and 3′ end of the NaChII gene but not the SCG10 gene in Rat-1 cells by ChIP analysis. Primers 1 and 2 (promoter) or primers 3 and 4 (coding region) were used for amplification of NaChII and SCG10 genomic sequences in ChIP analysis. (B) shows that MeCP2 complexes with endogenous CoREST protein from Rat-1 nuclear extracts. High-salt nuclear extracts prepared from Rat-1 cells were applied to antibody-agarose containing either preimmune (Preimmune) IgG or affinity-purified antibody to CoREST (anti-CoREST) and immunoprecipitated material analyzed with the use of antibodies to MeCP2. (C) compares the derepression of NaChII and sustained repression of SCG10 genes in Rat-1 cells after transient expression of the methyl DNA-binding domain of MeCP2 (MeCP2MDB) and holo-MeCP2 (MeCP2WT) in control cells or TSA-treated (for 6 hours) cells. NaChII gene expression is not affected by TSA treatment but is derepressed in the presence of MeCP2MBD. SCG10 gene expression is derepressed in the presence of TSA and the dominant negative MeCP2MBD has no effect on its expression. Constitutively expressed GAPDH gene was used as a control. (D) gives a time course for derepression of the NaChII gene in Rat-1 cells transiently expressing MeCP2MBD. 14 hours post-transfection of Rat-1 cells with constructs encoding MeCP2MBD reveals a relief of repression for NaChII but not SCG10. (E) is a representative ChIP experiment from Rat-1 cells using PCR primers designed for the NaChII and SCG10 gene promoters with specific antibodies to MeCP2, HP1, dimethyl-K9 H3, or preimmune IgG. The NaChII promoter appears to recruit MeCP2, HP1 and contains H3 histones methylated at lysine 9. (F) shows that the MeCP2-associated histone methyltransferase specifically methylates histone H3 but not H4 with the use of equal amounts of transiently expressed HA-tagged wild-type (Wt HA-MeCP2) and mutant (HA-MeCP2MBD) MeCP2 in Rat-1 cells. (G) demonstrates that SUV39H1 associated with holo-(wtHA-MeCP2) but not the mutant HA-tagged form of MeCP2 upon immunoprecipitation from whole cell extracts with mouse antibodies to HA (IP:anti-HA); Western blots were developed with rabbit IgG to SUV39H1 (antiSUV39H1) (top). Extracts precipitated with the use of rabbit IgG to SUV39H1 (IP: antiSUV39H1) were analyzed by Western blotting with mouse IgG to HA (anti-HA) to detect HA-tagged MeCP2. (H) is a two-stage immunoprecipitation ChIP demonstrating mutual occupancy of the NaChII gene promoter by MeCP2 and SUV39H1 proteins in Rat-1 cells.

6.3 Silencing of the Chromosomal Interval q22-32 on Rat Chromosome 3 in a REST/CoREST-dependent Manner

Chromosomal interval silencing has been demonstrated to occur for a number of different genomic regions including the centromere, at mating-type loci, and during X chromosome inactivation (Gregory et al., 2001, Mol. Cell. Biol., 21: 5426-31; Heard et al., 2001, Cell, 107: 727-32; Rea et al., 2000, Nature, 406: 593-98; Mechtler et al., 2000, Nature, 406: 593-98; Rice et al., 2001, Curr. OPin. Cell Biol., 13: 263-68; Noma et al., 2001, Science, 293: 1150-55; Bannister et al., 2001, Nature, 410: 120-25; Litt et al., 2001, Science, 293: 2453-58; Lachner et al., 2001, Nature, 410: 116-121). To determine whether a similar effect was observed with respect to REST target gene silencing expression studies were performed on REST target genes and adjacent loci in the chromosomal interval 3q22-32. FIG. 3 demonstrates the expression of particular loci within this region in the wild-type context as in the presence of TSA, 5AzaC and three independent dominant-negative constructs. (A) is an analysis of the expression of SMARCE and OTOF genes in Rat-1 cells treated with TSA and 5AzaC, dominant-negative MeCP2, REST/NRSF, or CoREST. Both genes require REST/NRSF for their repression, but overexpression of CoRESTRID or MeCP2MBD caused derepression of SMARCe in Rat-1 cells, whereas OTOF is reactivated only in the TSA-challenged cells. (B) shows derepression of a silent locus on rat Ch3. Expression profiling for wild-type and TSA- or 5AzaC-treated Rat-1 cells or Rat-1 cells transiently expressing RESTDBD, MeCP2MDB, and CoRESTRID was performed using RT-PCR. Treatment of Rat-1 cells with TSA did not alter the basal level of expression of genes within the putative REST/NRSF gene interval but did cause activation of the NeuroD1 gene. However, a derepression of all tested genes in the interval flanked by the NaChII and M4 REST/NRSF target genes was observed when cells were treated with 5AzaC. Genes containing binding sites for REST/NRSF in their promoters (NaChII, GAD1, and M4) are labeled with an asterisk (*).

6.4 Association of REST, CoREST and MeCP2 Proteins with the 3g22-32 Genomic Interval

Given the extent of silencing along the q22-32 interval of chromosome 3 and the derepression of this interval in the presence of 5AzaC as well as in the presence of dominant negative forms of REST, MeCP2 and CoREST it was necessary to confirm the presence of these proteins in their association with the interval. FIG. 4 is a compendium of ChIP results revealing the association of REST, CoREST and MeCP2 with interval 3q22-32. (A) is an analysis of the endogenous promoters of NaChII, NaChIII, GAD1, M4, HoxD9, and a 3′ coding region of NaChII genes by ChIP. Experiments were performed in wild-type Rat-1 cells and Rat-1 cells challenged by TSA or 5AzaC treatment. Positions of PCR products obtained by the amplification of genomic segment (G) and internal standards (I) are indicated. The results indicate that REST/NRSF and CoREST were associated only with the promoters of REST/NRSF-regulated genes and not detected on the 3′ end of the NaChII gene or on the promoters of the NaChIII or HoxD9 genes. In contrast, MeCP2 was present throughout the interval on the six tested genomic segments, but was released upon treatment with the demethylating agent 5AzaC. (B) reveals specific H3 modification at Lys9 (antiKm9H3) within chromosomal region 3q22-32. ChIP experiments were performed with antibodies to dimethyl-K9 H3 and dimethyal-K4 H3 across and outside of the silent interval. As is evidenced, only genes within the silenced interval contain methylated lysine-9 residues on histone H3.

6.5 CoREST-dependent gene silencing

Recruitment of the co-repressor CoREST to REST/NRSE gene targets appears to act as a molecular beacon for the silencing machinery, including MeCP2, SUV39H1, and HP1, to propagate and maintain a methyl CpG-dependent silent state across specific chromosomal intervals, including genes that do not contain REST/NRSF-binding sites. FIG. 5 illustrates this point via an analysis of the NaChII gene promoter in vivo by ChIP. (A) is a ChIP analysis of the NaChII promoter, using murine liver and heart tissues and antibodies specific to CoREST, MeCP2, and Km9H3. These data indicate the presence of MeCP2, CoREST and methylated lysine-9 residues on histone H3. (B) depicts a model of REST/CoREST-dependent silencing of a chromosomal interval, showing that binding of REST and specific CpG methylation events permit recruitment of CoREST and subsequent assembly and spreading of silencing machinery.

| | | |
|---|---|---|
| 5935811 | August 1999 | Anderson et al. |
| 6184211 | February 2001 | Szyf, M. |
| 6270990 | August 2001 | Anderson et al. |
| 6511808 | January 2003 | Wolffe et al. |
| 6555329 | April 2003 | Jenuwein et al. |

### Other References

Amir et al., “Rett syndrome is caused by mutations in X-linked MECP2, encoding methyl-CpG-binding protein 2”, 1999, Nature Genet., 23: 185-90.

Ballas et al., “Regulation of neuronal traits by a novel transcriptional complex”, 2001, Neuron, 31: 353-58.

Bannister et al., “Selective recognition of methylated lysine 9 on histone H3 by the HP1 chromo domain”, 2001, Nature, 410: 120-25.

Bell et al., “Methylation of a CTCF-dependent boundary controls imprinted expression of the Igf2 gene”, 2000, Nature, 405: 482-87.

Bird, A., “DNA methylation patterns and epigenetic memory”, 2002, Genes Dev., 16(1):6-21.

Brandies et al., “Dynamics of DNA methylation during development”, 1993, Bioessays, 15: 709-14.

Breiling et al., “General transcription factors bind promoters repressed by Polycomb group proteins”, 2001, Nature, 412(6847): 651-5.

Cao et al., “Role of Histone H3 Lysine 27 Methylation in Polycomb-Group Silencing”, 2002, Science, 298(5595): 1039-43.

Chen et al., 2001, “Deficiency of methyl-CpG binding protein-2 in CNS neurons results in a Rett-like phenotype in mice”, Nature Genet., 27: 327-332.

Chong et al., “REST: a mammalian silencer protein that restricts sodium channel gene expression to neurons”, 1995, Cell, 80: 949-957.

Cosma et al., “Ordered recruitment of transcription and chromatin remodeling factors to a cell cycle- and developmentally regulated promoter”, 1999, Cell, 97(3): 299-311.

Dhordain et al., “The LAZ3(BCL-6) oncoprotein recruits a SMRT/mSIN3A/histone deacetylase containing complex to mediate transcriptional repression”, 1998, Nucleic Acids Res., 26(20): 4645-51.

Gregory et al., “DNA methylation is linked to deacetylation of histone H3, but not H4, on the imprinted genes Snrpn and U2af1-rs1 ”, 2001, Mol. Cell. Biol., 21: 5426-31.

Hakimi et al., “A core-BRAF35 complex containing histone deacetylase mediates repression of neuronal-specific genes”, 2002, Proc. Natl. Acad. Sci. U.S.A., 99: 7420-24.

Heard et al., “Methylation of histone H3 at Lys-9 is an early mark on the X chromosome during X inactivation”, 2001, Cell, 107: 727-32.

Humphrey et al., “Stable histone deacetylase complexes distinguished by the presence of SANT domain proteins CoREST/kiaa0071 and Mta-L1”, 2001, J. Biol. Chem., 276:6817-22.

Jenuwein et al., “Translating the histone code”, 2001, Science, 293: 1074-79.

Jepsen et al., “Combinatorial roles of the nuclear receptor corepressor in transcription and development”, 2000, Cell, 102: 753-58.

Jones et al., “The fundamental role of epigenetic events in cancer”, 2002, Nature Rev. Genet., 3: 415-420.

Kokura et al., “The Ski protein family is required for MeCP2-mediated transcriptional repression”, 2001, J. Biol. Chem., 276: 34115-20.

Lachner et al., “Methylation of histone H3 lysine 9 creates a binding site for HP1 proteins”, 2001, Nature, 410: 116-121.

Lewis et al., “Purification, sequence, and cellular localization of a novel chromosomal protein that binds to methylated DNA”, 1992, Cell, 69: 905-10.

Litt et al., “Correlation between histone lysine methylation and developmental changes at the chicken beta-globin locus”, 2001, Science, 293: 2453-58.

Lorincz et al., “Methylation-mediated proviral silencing is associated with MeCP2 recruitment and localized histone H3 deacetylation”, 2001, Mol. Cell Biol., 21: 7913-18.

Lunyak et al., “Co-repressor-dependent silencing of chromosomal regions encoding neuronal genes”, 2002, Science, 298: 1747-51.

Mechtler et al., “Regulation of chromatin structure by site-specific histone H3 methyltransferases”, 2000, Nature, 406: 593-98.

Mieda et al., “Expression of the rat m4 muscarinic acetylcholine receptor gene is regulated by the neuron-restrictive silencer element/repressor element 1”, 1997, J. Biol. Chem., 272: 5854-59.

Mori et al., “A cell type-preferred silencer element that controls the neural-specific expression of the SCG10 gene”, 1990, Neuron, 4: 583-88.

Nagy et al., “Nuclear receptor repression mediated by a complex containing SMRT, mSin3a, and histone deacetylase”, 1997, Cell, 89(3): 373-80.

Nakayama et al., “A chromodomain protein, Swi6, performs imprinting functions in fission yeast during mitosis and meiosis”, 2000, Cell, 101: 307-12.

Nakayama et al., “Role of histone H3 lysine 9 methylation in epigenetic control of heterochromatin assembly”, 2001, Science, 292: 110-14.

Naruse et al., “Neural restrictive silencer factor recruits mSin3 and histone deacetylase complex to repress neuron-specific target genes”, 1999, Proc. Natl. Acad. Sci. U.S.A., 96: 13691-95.

Noma et al., “Transitions in distinct histone H3 methylation patterns at the heterochromatin domain boundaries”, 2001, Science, 293: 1150-55.

Pevzner et al., 2000, Intelligent Systems in Molecular Biology, AAAI Press, La Jolla, Calif., 269-78.

Razin et al., “DNA methylation patterns. Formation and function”, 1989, Biochim. Biophys. Acta, 782: 331-36.

Rea et al., “Regulation of chromatin structure by site-specific histone H3 methyltransferases”, 2000, Nature, 406: 593-98.

Rice et al., “Histone methylation versus histone acetylation: new insights into epigenetic regulation”, 2001, Curr. Opin. Cell Biol., 13: 263-68.

Schoenherr et al., “The neuron-restrictive silencer factor (NRSF): a coordinate repressor of multiple neuron-specific genes”, 1995, Science, 267: 1360-65.

Schoenherr et al., “Identification of potential target genes for the neuron-restrictive silencer factor” 1996, Proc. Natl. Acad. Sci. U.S.A., 93: 9881-9886.

Siegfried et al., “DNA methylation represses transcription in vivo”, 1999, Nature Genet., 22: 203-208.

Tate et al., 1993, “Effects of DNA methylation on DNA-binding proteins and gene expression”, Curr. Opin. Genet. Dev., 3: 226-231.

Tweedie et al., “Methylation of genomes and genes at the invertebrate-vertebrate boundary”, 1997, Mol. Cell. Biol., 17: 1469-74.

You et al., “CoREST is an integral component of the CoREST-human histone deacetylase complex”, 2001, Proc. Natl. Acad. Sci. U.S.A., 98: 1454-59.

Yu et al., “Transcriptional repression by blimp-1 (PRDI-BF1) involves recruitment of histone deacetylase”, 2000, Mol. Cell Biol., 20(7): 2592-603.

Wood et al., “Neural specific expression of the m4 muscarinic acetylcholine receptor gene is mediated by a RE1/NRSE-type silencing element”, 1996, J. Biol. Chem., 271:14221-26.

What is claimed is:

1. A biochemical complex, referred to as Complex A, which has the capacity to drive long-term gene silencing containing:
A. REST/NRSF
B. CoREST
C. MeCP2
D. SUV39H1

2. The individual components of Complex A according to claim 1, including:
A. REST/NRSF
B. CoREST
C. MeCP2
D. SUV39H1

3. Other as yet undefined biochemical members of Complex A according to claim 1.

4. Truncated forms of REST/NRSF according to claim 2 which act in a dominant negative fashion to abrogate REST/NRSF activity.

5. Truncated forms of CoREST according to claim 2 which act in a dominant negative fashion to abrogate CoREST function.

6. Truncated forms of MeCP2 according to claim 2 which act in a dominant negative fashion to abrogate MeCP2 function.

7. As yet undiscovered methyltransferases identified as interacting with REST/NRSF-containing biochemical complexes according to claim 1.

8. As yet undiscovered methyltransferases identified as interacting with CoREST-containing biochemical complexes according to claim 1.

9. As yet undiscovered methyltransferases identified as interacting with MeCP2-containing biochemical complexes according to claim 1.

10. As yet undiscovered cofactors identified as interacting with REST/NRSF-containing biochemical complexes according to claim 1.

11. The REST/NRSF consensus DNA binding site.

12. Variants of the REST/NRSF consensus DNA binding site according to claim 11 that recruit REST/NRSF and REST/NRSF—containing biochemical complexes.

13. A bioinformatics-based approach to identifying REST/NRSF transcriptional target genes based upon the presence of one or more REST/NRSF DNA binding sites according to claim 12.

14. A nonbioinformatics-based approach to identifying REST/NRSF transcriptional target genes based upon the presence of one or more REST/NRSF DNA binding sites according to claim 12.

15. The positional organization of REST/NRSF DNA binding sites according to claim 12 within target genes.

16. REST/NRSF target genes identified as interacting with REST/NRSF.

17. The effect of cell division suppression through the use of dominant negative constructs which interfere with REST/NRSF function according to claim 1.

18. The effect of apoptosis through the use of dominant negative constructs which interfere with REST/NRSF function according to claim 1.

19. Molecules designed to interact with and enhance or abrogate the function of Complex A or the function of individual members of Complex A according to claim 1.

20. A spreading effect which results in specific chromosomal interval silencing according to claim 1.
